(19) United States
(12) Patent Application Publication
(10) Pub. No.: US 2010/0279408 A1
(43) Pub. Date: Nov. 4, 2010

(54) POLYMERIC SHORT INTERFERING RNA CONJUGATES

(75) Inventor: Hong Zhao, Edison, NJ (US)

Correspondence Address: LUCAS & MERCANTI, LLP, 475 PARK AVENUE SOUTH, 15TH FLOOR, NEW YORK, NY, 10016, US

(73) Assignee: ENZON PHARMACEUTICALS, INC., Bridgewater, NJ (US)

(21) Appl. No.: 12/445,814

(22) PCT Filed: Nov. 27, 2007
(86) PCT No.: PCT/US07/85616

§ 371 Date: Apr. 16, 2009

Related U.S. Application Data
(60) Provisional application No. 60/911,739, filed on Apr. 13, 2007.
(60) Provisional application No. 60/861,382, filed on Nov. 27, 2006.

(51) Int. Cl. C07H 21/02; C12N 5/00

(52) U.S. Cl. 435/375; 536/24.5

(57) ABSTRACT

The present invention provides polymeric siRNA conjugates. Methods for down-regulation of gene expression in vivo and in vitro and for inhibition of the growth of cancer cells using the conjugates are also disclosed.

### CROSS-REFERENCE TO RELATED APPLICATION

This application claims the benefit of priority from U.S. Provisional Patent Application Ser. No. 60/861,382 filed Nov. 27, 2006 and 60/911,739 filed Apr. 13, 2007, the contents of each of which are incorporated herein by reference.

### BACKGROUND OF THE INVENTION

Small interfering RNA or short interfering RNA (siRNA) is a double stranded RNA molecule. siRNA inteferes with a gene expression and induces degradation of mRNA expressed from the gene. Thus, RNA interference mediated by siRNA has emerged as a potentially powerful anticancer therapeutic agent over the past few years. The development of short interfering RNA (siRNA) as therapeutics has, however, been limited due to their inefficient delivery, poor stability and suboptimal pharmacokinetic (PK) profile.

Some proposals have been made to overcome the hurdle to use siRNAs as therapeutics. One of such attempts to improve delivery and enhance cellular uptake of siRNA is directed to utilizing liposomes and nanoparticles. See Yano et al., Clinical Cancer Research, 10:7721-7726; Bartlett et al., Bioconjugate Chem., 18:456-468. Other attempts include uses of polymers such as hyaluronic acid nanogels. See Lee et al., Journal of Controlled Release. “Target-specific Intracellular delivery of siRNA Using Degradable Hyaluronic Acid Nanogels”, in press, 2007. Alternatively, in other attempts to improve delivery of siRNA, transfection agents are used in attempts to improve delivery of siRNA. See Wang et al., Journal of Biochemical and Biophysical Methods, “An Intracellular Delivery Method For siRNA By An Arginine-rich Peptide”, in press, 2007. Transfections are not, however, desired in uses of therapeutic siRNA in vivo. Presently, direct intra-compartmental injection is still the major route of administration. In spite of the attempts and advances, there continues to be a heed to provide improved delivery systems of siRNA. The present invention addresses this need.

### SUMMARY OF THE INVENTION

In order to overcome the above problems and improve the technology for siRNA delivery, there are provided new polymeric siRNA delivery systems.

In one aspect of the present invention, there are provided siRNA conjugates of Formula (I):

A-R₁—(R₂)ₑ—R₃

wherein

A includes a capping group or

R′₃—(Rⁱ₂)_{e′}—;
R₁ includes a substantially non-antigenic water-soluble polymer;

R₂ and R′₂ are independently selected releasable or permanent linkers or a combination thereof;

R₃ and R′₃ are the same or different siRNA-containing moiety; and

(e) and (e′) are the same or different positive integers, preferably 1 or 2.

Preferably, R₂ and, when present R′₂ are linked to the sense strand of the siRNA-containing moiety.

In one preferred aspect of the invention, the siRNA-containing: moieties are attached to the polymeric portion of the compounds described herein via releasable linkers. Alternatively, the releasable linkers are preferably intracellular labile linkers and/or acid labile linkers.

In another preferred aspect of the present invention, there are provided methods of inhibiting gene expression such as for BCL2. The methods include contacting human cells such as cancer cells or tissues with the PEG-siRNA conjugates described herein. The conjugates mediate down-regulation of BCL2 mRNA or protein in the cells being treated in human cells and tissues.

In yet another preferred aspect, the treatment With the PEG-siRNA conjugates described herein allow down-modulation of BCL2 mRNA and the attendant benefits associated therewith in the treatment of malignant disease such as inhition of the growth of cancer cells. Such therapies can be carried out as a single treatment or as apart of combination therapy with one or more useful and/or approved treatments.

One advantage of the present invention is that the customized releasable PEG-linker technology provides a method for in vivo administration of siRNA molecules. This delivery technology allows enhanced biostability and therapeutic efficacy of siRNA.

The siRNA conjugates described herein stabilize siRNA in biological fluids. Without being bound by any theory, it is believed that the conjugates enhance the stability of siRNA at least in part through an increase in the resistance towards nucleases. The polymeric siRNA conjugates are also stable under buffer conditions. Moreover, because they are part of a conjugate, the siRNAs are not prematurely excreted from the body.

Another advantage is that the conjugates described herein allow for modulating of the pharmacokinetic properties of siRNA. The release rates/sites of siRNA from the polymeric conjugates can be modified. The siRNAs attached to the polymers described herein can be released at predetermined and predictable rates, thus allowing the artisan to achieve a desired bioavailability of therapeutic siRNA. The site of release of the negatively-charged therapeutic siRNA can be also modified, i.e. release at different compartments of cells. Thus, the polymeric delivery systems described herein allow sufficient amounts of the therapeutic siRNA to be selectively available at the desired target area, i.e. cytoplasm. In particular, because, the siRNA is conjugated to the polymer via the sense strand, the antisense strand of siRNA molecules can dissociate from the siRNA duplex in acidic environment of cytoplasm and induce the desired RNA interference. The antisense strand is completely unencumbered by the polymer conjugation. The temporal and spatial modifications alone and in combination of release of the therapeutic agents are advantageous for treatment of disease.

A further advantage of the present invention is that the conjugates described herein allow cellular uptake and specific mRNA down regulation in cancer cells in the absence of transfection agents. This is a significant advantage over prior art technologies and thus significantly simplifies treatment regimens. This technology can be applied to the in vivo administration of therapeutic siRNA.

Other and further advantages will be apparent from the following description.

For purposes of the present invention, the term “residue” shall be understood to mean that portion of a compound, to which it refers, i.e. PEG, oligonucleotide, etc. that remains after it has undergone a substitution reaction with another compound.

For purposes of the present invention, the term “polymeric residue” or “PEG residue” shall each be understood to mean that portion of the polymer or PEG which remains after it has undergone a reaction with other compounds, moieties, etc.

For purposes of the present invention, the term “alkyl” as used herein refers to a saturated aliphatic hydrocarbon, including straight-chain, branched-chain, and cyclic alkyl groups. The term “alkyl” also includes alkyl-thio-alkyl, alkoxyalkyl, cycloalkylalkyl, heterocycloalkyl, C₁₋₆ hydrocarbonyl, groups. Preferably, the alkyl group has 1 to 12 carbons. More preferably, it is a lower alkyl of from about 1 to 7 carbons, yet more preferably about 1 to 4 carbons. The alkyl group can be substituted or unsubstituted. When substituted, the substituted group(s) preferably include halo, oxy, azido, nitro, cyano, alkyl, alkoxy, alkyl-thio, alkyl-thio-alkyl, alkoxyalkyl, alkylamino, trihalomethyl, hydroxyl, mercapto, hydroxy, cyano, alkylsilyl, cycloalkyl, cycloalkylalkyl, heterocycloalkyl, heteroaryl, alkenyl, alkynyl, C₁₋₆ hydrocarbonyl, aryl, and amino groups.

For purposes of the present invention, the term “substituted” as used herein refers to adding or replacing one or more atoms contained within a functional group or compound with one of the moieties from the group of halo, oxy, azido, nitro, cyano, alkyl, alkoxy, alkyl-thio, alkyl-thio-alkyl, alkoxyalkyl, alkylamino, trihalomethyl, hydroxyl, mercapto, hydroxy, cyano, alkylsilyl, cycloalkyl, cycloalkylalkyl, heterocycloalkyl, heteroaryl, alkenyl, alkynyl, C₁₋₆ hydrocarbonyl, aryl, and amino groups.

The term “alkenyl” as used herein refers to groups containing at least one carbon-carbon double bond, including straight-chain, branched-chain, and cyclic groups. Preferably, the alkenyl group has about 2 to 12 carbons. More preferably, it is a lower alkenyl of from about 2 to 7 carbons, yet more preferably about 2 to 4 carbons. The alkenyl group can be substituted or unsubstituted. When substituted the substituted group(s) preferably include halo, oxy, azido, nitro, cyano, alkyl, alkoxy, alkyl-thio, alkyl-thio-alkyl, alkoxyalkyl, alkylamino, trihalomethyl, hydroxyl, mercapto, hydroxy, cyano, alkylsilyl, cycloalkyl, cycloalkylalkyl, heterocycloalkyl, heteroaryl, alkenyl, alkynyl, C₁₋₆ hydrocarbonyl, aryl, and amino groups.

The term “alkynyl” as used herein refers to groups containing at least one carbon-carbon triple bond, including straight-chain, branched-chain, and cyclic groups. Preferably, the alkynyl group has about 2 to 12 carbons. More preferably, it is a lower alkynyl of from about 2 to 7 carbons, yet more preferably about 2 to 4 carbons. The alkynyl group can be substituted or unsubstituted. When substituted the substituted group(s) preferably include halo, oxy, azido, nitro, cyano, alkyl, alkoxy, alkyl-thio, alkyl-thio-alkyl, alkoxyalkyl, alkylamino, trihalomethyl, hydroxyl, mercaplo, hydroxy, cyano, alkylsilyl, cycloalkyl, cycloalkylalkyl, heterocycloalkyl, heteroaryl, alkenyl, alkynyl, C₁₋₆ hydrocarbonyl, aryl, and amino groups. Examples of “alkynyl” include propargyl, propyne, and 3-hexyne.

The term “aryl” as used herein refers to an aromatic hydrocarbon ring system containing at least one aromatic ring. The aromatic ring can optionally be fused or otherwise attached to other aromatic hydrocarbon rings or non-aromatic hydrocarbon rings. Examples of aryl groups include, for example, phenyl, naphthyl, 1,2,3,4-tetrahydronaphthalene and biphenyl. Preferred examples of aryl groups include phenyl and naphthyl.

The term “cycloalkyl” as used herein refers to a C₃₋₈ cyclic hydrocarbon. Examples of cycloalkyl include cyclopropyl, cyclobutyl, cyclopentyl, cyclohexyl, cycloheptyl and cyclooctyl.

The term “cycloalkenyl” as used herein refers to a C₃₋₈ cyclic hydrocarbon containing at least one carbon-carbon double bond. Examples of cycloalkenyl include cyclopentenyl, cyclopentadienyl, cyclohexenyl, 1,3-cyclohexadienyl, cycloheptenyl, cycloheptatrienyl, and cyclooctenyl.

The term “cycloalkylalkyl” as used herein refers to an alklyl group substituted with a C₃₋₈ cycloalkyl group. Examples of cycloalkylalkyl groups include cyclopropylmethyl and cyclopentylethyl.

The term “alkoxy” as used herein refers to an alkyl group of indicated number of carbon atoms attached to the parent molecular moiety through an oxygen bridge. Examples of alkoxy groups include, for example, methoxy, ethoxy, propoxy and isopropoxy.

An “alkylaryl” group as used herein refers to an aryl group substituted with an alkyl group.

An “aralkyl” group as used herein refers to an alkyl group substituted with an aryl group.

The term “alkoxyalkyl” group as used herein refers to an alkyl group substituted with an alkloxy group.

The term “alkyl-thio-alkyl” as used herein refers to an alkyl-S-alkyl thioether, for example methylthiomethyl or methylthioethyl.

The term “amino” as used herein refers to a nitrogen containing group as is known in the art derived from ammonia by the replacement of one or more hydrogen radicals by organic radicals. For example, the terms “acylamino” and “alkylamino” refer to specific N-substituted organic radicals with acyl and alkyl substituent groups respectively.

The term “alkylcarbonyl” as used herein refers to a carbonyl group substituted with alkyl group.

The terms “halogen” or “halo” as used herein refer to fluorine, chlorine, bromine, and iodine.

The term “heterocycloalkyl” as used herein refers to a non-aromatic ting system containing at least one heteroatom selected from nitrogen, oxygen, and sulfur. The heterocycloalkyl ring can be optionally fused to or otherwise attached to other heterocycloalkyl rings and/or non-aromatic hydrocarbon rings. Preferred heterocycloalkyl groups have from 3 to 7 members. Examples of heterocycloalkyl groups include, for example, piperazine, morpholine, piperidine, tetrahydrofuran, pyrrolidine, and pyrazole. Preferred heterocycloalkyl groups include piperidinyl, piperazinyl, morpholinyl, and pyrolidinyl.

The term “heteroaryl” as used herein refers to an aromatic ring system containing at least one heteroatom selected from nitrogen, oxygen, and sulfur. The heteroaryl ring can be fused or otherwise attached to one or more heteroaryl rings, aromatic or non-aromatic hydrocarbon rings or heterocycloalkyl rings. Examples of heteroaryl groups include, for example, pyridine, furan, thiophene, 5,6,7,8-tetrahydroisoquinoline and pyrimidine. Preferred examples of heteroaryl groups include thienyl, benzothienyl, pyridyl, quinolyl, pyrazinyl, pyrimidyl, imidazolyl, benzimidazolyl, furanyl, benzofuranyl, thiazolyl, benzothiazolyl, isoxazolyl, oxadiazolyl, isothiazolyl, benzisothiazolyl, triazolyl, tetrazolyl, pyrrolyl, indolyl, pyrazolyl, and benzopyrazolyl.

The term “heteroatom” as used herein refers to nitrogen, oxygen, and sulfur.

In some embodiments, substituted alkyls include carboxyalkyls, aminoalkyls, dialkylaminos, hydroxyalkyls and mercaptoalkyls; substituted alkenyls include carboxyalkenyls, aminoalkenyls, dialkenylaminos, hydroxyalkenyls and mercaptoalkenyls; substituted alkynyls include carboxyalkynyls, amino alkynyls, dialkynylaminos, hydroxyalkynyls and mercaptoalkynyls; substituted cycloalkyls include moieties such as 4-chlorocyclohexyl; aryls include moieties such as napthyl; substituted aryls include moieties such as 3-bromo phenyl; aralkyls include moieties such as tolyl; heteroalkyls include moieties such as ethylthiophene; substituted heteroalkyls include moieties such as 3-methoxy-thiophene; alkoxy includes moieties such as methoxy; and phenoxy includes moieties such as 3-nitrophenoxy. Halo shall be understood to include fluoro, chloro, iodo and bromo.

For purposes of the present invention, “positive integer” shall be understood to include an integer equal to or greater than 1 and as will be understood by those of ordinary skill to be within the realm of reasonableness by the artisan of ordinary skill, i.e., preferably from 1 to about 10, more preferably 1 or 2 in some embodiments.

For purposes of the present invention, the term “linked” shall be understood to include covalent (preferably) or noncovalent attachment of one group to another, i.e., as a result of a chemical reaction.

The terms “effective amounts” and “sufficient amounts” for purposes of the present invention shall mean an amount which achieves a desired effect, or therapeutic effect as such effect is understood by those of ordinary skill in the art.

### BRIEF DESCRIPTION OF THE DRAWINGS

FIG. 1 schematically illustrates methods of synthesis described in Example 1.

FIG. 2 schematically illustrates methods of synthesis described in Example 2.

FIG. 3 schematically illustrates methods of synthesis described in Example 3.

FIG. 4 shows PEG-siRNA stability described in Example 5.

FIG. 5 shows in vitro BCL2 expression study described in Example 6.

FIG. 6 shows in vivo BCL2 expression study described in Example 7.

FIG. 7 shows the PK study described in Example 9.

### DETAILED DESCRIPTION OF THE INVENTION

### A. Overview

In one aspect of the present invention, there are provided siRNA conjugates of Formula (I):

A-R₁—(R₂)ₑ—R₃

wherein

A includes a capping group or

R′₃—(R′₂)_{e′}—;

R₁ includes a substantially non-antigenic water-soluble polymer;

R₂ and R′₂ are independently selected releasable or permanent, linkers or a combination thereof;

R₃ and R′₃ are the same or different siRNA-containing moiety; and

(e) and (e′) are the same or different positive integers, preferably 1 or 2.

Preferably, R₂ and, when present R′₂ are linked to the sense strand of the siRNA-containing moiety.

In one aspect of the present invention, the conjugates described herein include the capping group such as H, NH₂, OH, CO₂H, C₁₋₆ alkoxy and C₁₋₆ alkyl. In one preferred aspect, the capping group includes CH₃ or CH₃O.

In another aspect of the present invention, the conjugates have the formula:

R′₃—(R′₂)_{e′}—R₁—(R₂)ₑ—R₃.

The polymers contemplated with this aspect can therefore include linear PEGs, bis-PEGs, U-PEG mid multi-arm PEGs.

In one preferred embodiment, the conjugates described herein can have the formula:

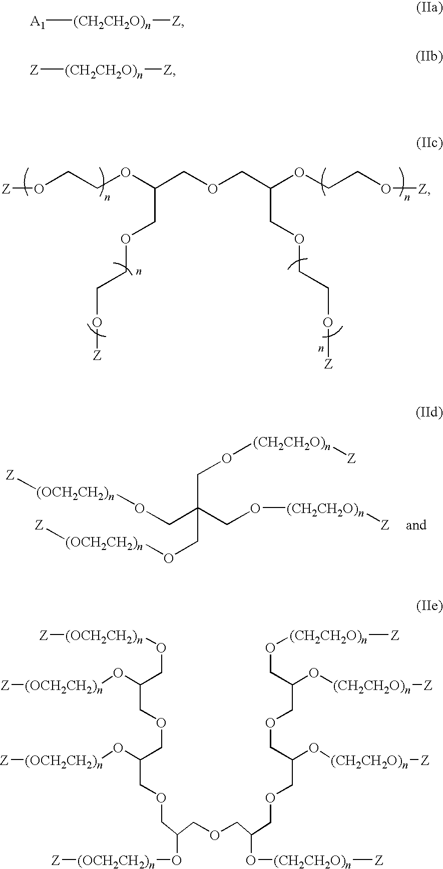

wherein

(n) is an integer from about 10 to about 2300, where the total molecular weight of the polymeric portion is from about 2,000 to about 100,000 Daltons;

A₁ includes a capping group such as H, NH₃, OH, CO₂H, C₁₋₆ alkoxy, C₁₋₆ alkyl, and C₁₋₆ alkyl substituted amines, preferably CH₃ or CH₃O;

one or more Z can be

—(R₂)ₑ—R₃; and

all other variables are previously defined. In alternative aspects, one or more of the Z groups can be other than —(R₂)ₑ—R₃ such as capping groups, i.e. H, OH, CH₃, OCH₃, or C₁₋₆ alkyl substituted amines such as n-butyl amine. Preferably, in the conjugates employing multi-arm polymers such as eight armed polymers, one Z group includes —(R₂)ₑ—R₃ and other Z groups include capping groups or functional groups.

In one preferred aspect of the invention, there are provided polymeric siRNA conjugates using releasable PEG (rPEG) linker technology. The siRNA-containing moieties are attached to the polymeric portion of the compounds described herein via releasable linkers preferably to the sense strand of the duplex. Among the releasable linkers can be benzyl elimination-based linkers, trialkyl lock-based linkers, bicine-based linkers, a disulfide bond, hydrazone-containing linkers and thiopropionate-containing linkers. Alternatively, the releasable linkers can be intracellular labile linkers, extracellular linkers or acid labile linkers. More preferably, the releasable linkers are intracellular labile linkers or acid labile linkers.

Alternatively, the siRNA can be attached to the polymer via the antisense strand using the techniques described with regard to the sense strand attachment. In this aspect, however, the linker selected for releasably joining the siRNA to the polymer should be one which facilitates release or generation of the antisense strand intracellularly. Such linkers include, for example, the acid labile linkers and/or intracellular labile linkers (i.e., disulfide group) described herein.

In another preferred aspect of the invention, polymeric siRNA conjugates with releasable linkers employ BCL2 siRNA. BCL2 protein is overexpressed in many types of tumors. Alternatively, a person of ordinary skill will appreciate that alternative suitable oncogenes having similar biological activity against cancer or other diseases can be employed in the polymeric siRNA conjugates.

In a more preferred aspect of the present invention, there are provided releasable PEG-siRNA conjugates in which the 5′-end of the sense strand of the siRNA duplex is linked to a C₆-amino tail for conjugating to PEG linkers.

### B. Substantially Non-Antigenice Polymers

Polymers employed in the compounds described herein are preferably water soluble polymers and substantially non-antigenic such as polyalkylene oxides (PAO's).

In one aspect of the invention, the compounds described herein can include a linear, terminally branched or multi-armed polyalkylene oxide. In some preferred embodiments of the invention, the polyalkylene oxide includes polyethylene glycol and polypropylene glycol

The polyalkylene oxide has an average molecular weight from about 2,000 to about 100,000 Daltons in most aspects of the invention. Preferably, the polymer can be from about 5,000 to about 60,000 Daltons, more preferably from about 20,000 to about 45,000. Yet more preferably, the polymer has a weight average molecular weight of about 30,000 Daltons. Other molecular weights are also contemplated so as to accommodate the needs of the artisan.

The polyalkylene oxide includes polyethylene glycols and polypropylene glycols. More preferably, the polyalkylene oxide includes polyethylene glycol (PEG). PEG is generally represented by the structure:

—O—(CH₂CH₃O)ₙ—

where (a) is an integer from about 10 to about 2,300, and is dependent on the number of polymer arms when multi-arm polymers are used. Alternatively, the polyethylene glycol (PEG) residue portion of the invention can be represented by the structure:

—Y₇₁—(CH₂CH₂O)ₙ—CH₂CH₂Y₇₁—,

—Y₇₁—(CH₂CH₂O)ₙ—CH₂C(═Y₂₂)—Y₇₁—,

—Y₇₁—C(═Y₇₂)—(CH₂)ₐ₂—Y₇₃—(CH₂CH₂O)ₙ—CH₂CH₂—Y₇₃—(CH₂)ₐ₂—C(═Y₇₂)—Y₇₁— and

—Y₇₁—(CR₇₁R₇₂)ₐ₂—Y₇₃—(CH₂)_{b2}—O—(CH₂CH₂O)ₙ—(CH₂)_{b2}—Y₇₃—(CR₇₁R₇₂)ₙ₂—Y₇₁—,

wherein:

Y₇₁ and Y₇₃ are independently O, S, SO, SO₂, NR₇₃ or a bond;

Y₇₂ is O, S, or NR₇₄;

R₇₁₋₇₄ are independently selected from among hydrogen, C₁₋₆ alkyl, C₂₋₆ alkenyl, C₂₋₆ alkynyl, C₃₋₁₉ branched alkyl, C₃₋₈ cycloalkyl, C₁₋₆ substituted alkyl, C₂₋₆ substituted alkenyl, C₂₋₆ substituted alkynyl, C₃₋₈ substituted cycloalkyl, aryl, substituted aryl, heteroaryl, substituted heteroaryl, C₁₋₆ heteroalkyl, substituted C₁₋₆ heteroalkyl, C₁₋₆ alkoxy, aryloxy, C₁₋₆ heteroalkoxy, heteroaryloxy, C₂₋₆ alkanoyl, arylcarbonyl, C₂₋₆ alkoxycarbonyl, aryloxycarbonyl, C₂₋₆ alkanoyloxy, arylcarbonyloxy, C₂₋₆ substituted alkanoyl, substituted arylcarbonyl, C₂₋₆ substituted alkanoyloxy, substituted aryloxycarbonyl, C₂₋₆ substituted alkanoyloxy and substituted arylcarbonyloxy;

(a2) and (b2) are independently zero or a positive integer, preferably zero or an integer from about 1 to about 6, and more preferably 1; and

(n) is an integer from about 10 to about 2300.

Branched or U-PEG derivatives are described in U.S. Pat. Nos. 5,643,575, 5,919,455, 6,113,906 and 6,566,506, the disclosure of each of which is incorporated herein by reference. A non-limiting list of such polymers corresponds to polymer systems (i)-(vii) with the following structures:

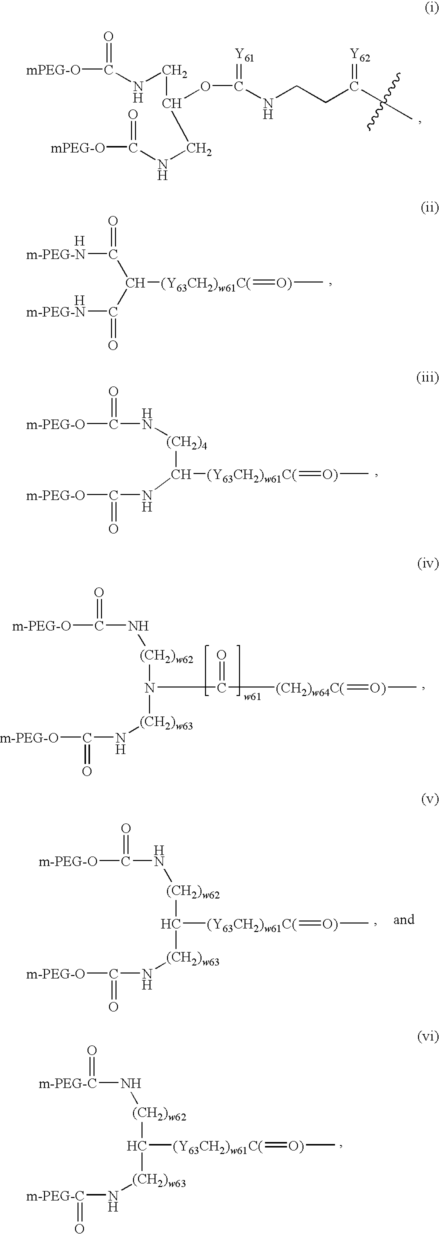

wherein:

Y₆₁₋₆₂ are independently O, S or NR₆₁;

Y₆₃ is O, NR₆₂, S, SO or SO₂

(w62), (w63) and (w64) are independently 0 or a positive integer, preferably zero or an integer from about 1 to about 3;

(w61) is 0 or 1;

mPEG is methoxy PEG
wherein PEG is previously defined and a total molecular weight of the polymer portion is from about 2,000 to about 100,000 Daltons; and

R₆₁ and R₆₂ are independently the same moieties which can be used for R₇₃.

In yet another aspect, the polymers include multi-arm PEG-OH or “star-PEG” products such as those described in NOF Corp. Drug Delivery System catalog, Ver. 8, April 2006, the disclosure of which is incorporated herein by reference. See also Shearwater Corporation's 2001 catalog “Polyethylene Glycol and Derivatives for Biomedical Application”, the disclosure of which is incorporated herein by reference. The multi-arm polymer conjugates contain four or more polymer arms and preferably four or eight polymer arms.

For purposes of illustration and not limitation, the multi-arm polyethylene glycol (PEG) residue can be

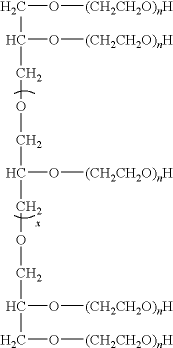

wherein:

(x) is zero and a positive integer, i.e. from about 0 to about 28; and

(n) is the degree of polymerization.

In one particular embodiment of the present invention, the multi-arm PEG has the structure:

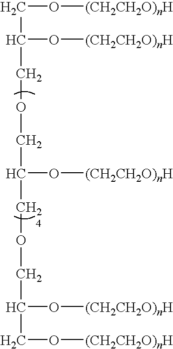

wherein (n) is a positive integer. In one preferred embodiment of the invention, the polymers have a total molecular weight of from about 5,000 Da to about 60,000 Da, and preferably from 20,000 Da to 45,000 Da.

In yet another particular embodiment, the multi-arm PEG has the structure:

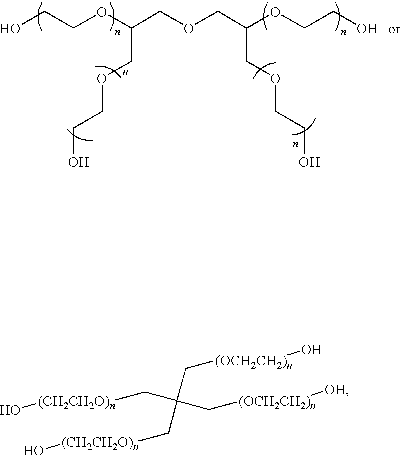

wherein (n) is a positive integer. In one preferred embodiment of the invention, the degree of polymerization for the multi-arm polymer (n) is from about 28 to about 350 to provide polymers having a total molecular weight of from about 5,000 Da to about 60,000 Da, and preferably from 12,000 Da to 45,000 Da. This represents the number of repeating units in the polymer chain and is dependent on the molecular weight of the polymer.

The polymers can be converted into a suitably activated polymer, using the activation techniques described in U.S. Pat. Nos. 5,122,614 or 5,808,096. Specifically, such PEG can be of the formula:

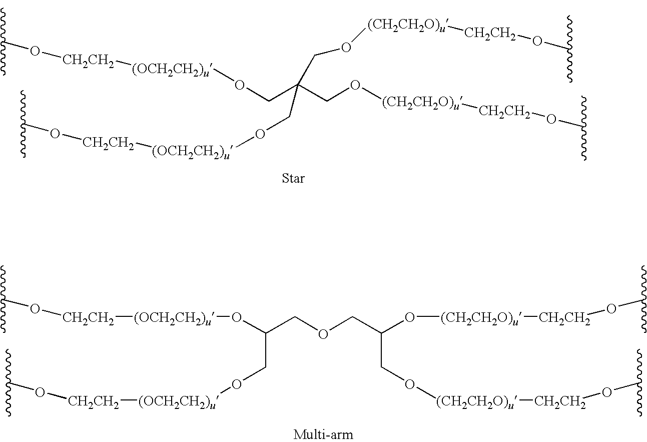

wherein:

(u′) is an integer from about 4 to about 455; and up to 3 terminal portions of the residue is/are capped with a methyl or other lower alkyl.

In some preferred embodiments, all four of the PEG arms can be converted to suitable activating groups, for facilitating attachment to aromatic groups. Such compounds prior to conversion include:

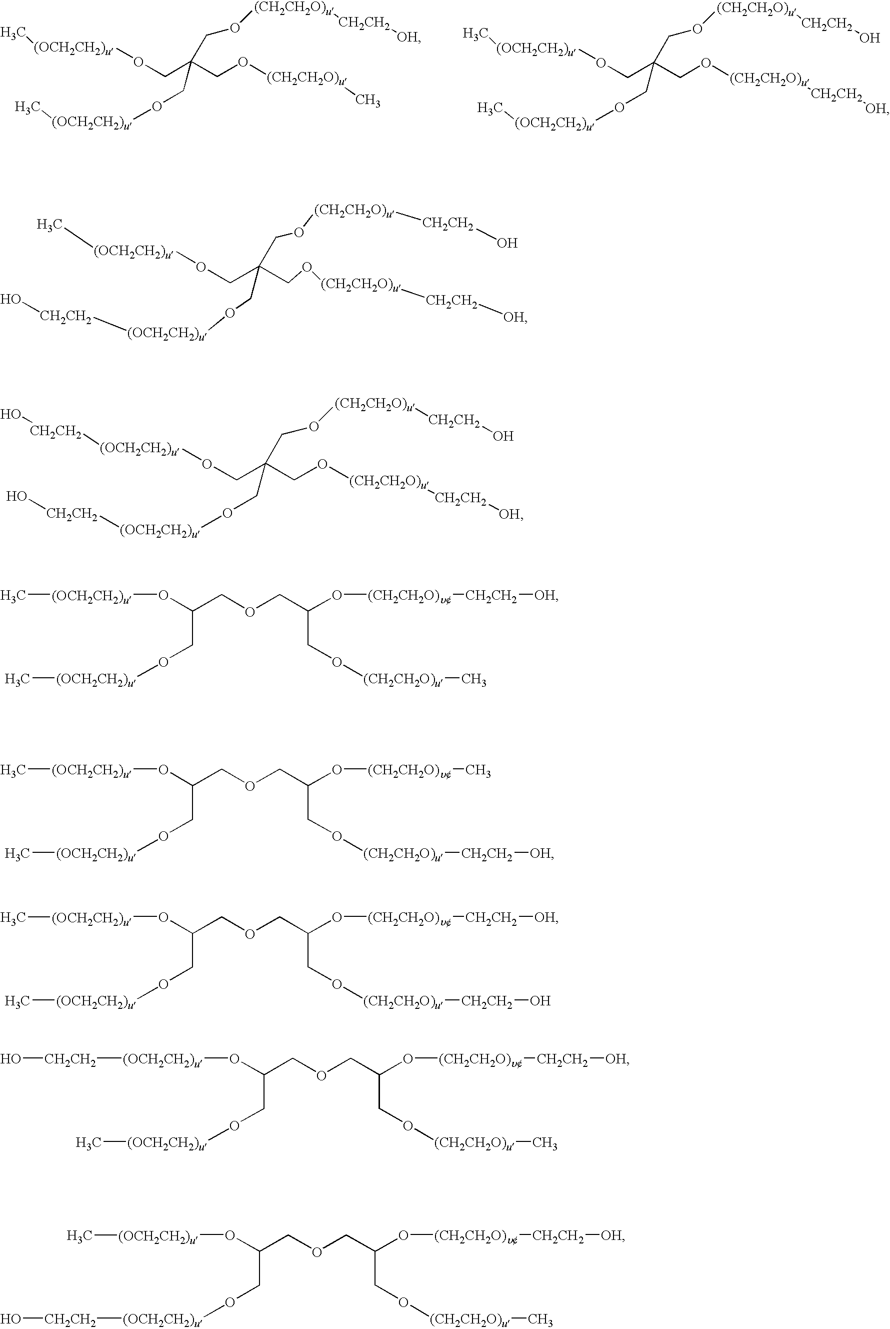

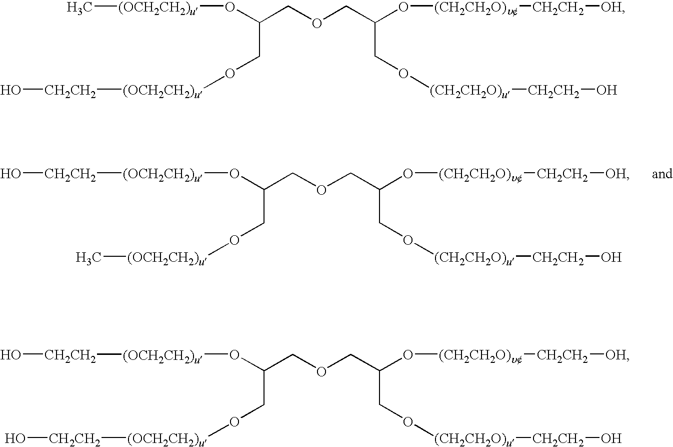

The polymeric substances included herein are preferably water-soluble at room temperature. A non-limiting list of such polymers include polyalkylene oxide homopolymers such as polyethylene glycol (PEG) or polypropylene glycols, polyoxyethylenated polyols, copolymers thereof and block copolymers thereof, provided that the water solubility of the block copolymers is maintained.

In a further embodiment, and as an alternative to PAO-based polymers, one or more effectively non-antigenic materials such as dextran, polyvinyl alcohols, carbohydrate-based polymers, hydroxypropylmethacrylamide (HPMA), polyalkylene oxides, and/or copolymers thereof can be used. See also commonly-assigned U.S. Pat. No. 6,153,655, the contents of which are incorporated herein by reference. It will be understood by those of ordinary skill that the same type of activation is employed as described herein as for PAO's such as PEG. Those of ordinary skill in the art will further realize that the foregoing list is merely illustrative and that all polymeric materials having the qualities described herein are contemplated. For purposes of the present invention, “substantially of effectively non-antigenic” means all materials understood in the art as being nontoxic and not eliciting an appreciable immunogenic response in mammals.

In some aspects, polymers having terminal amine groups can be employed to make the compounds described herein. The methods of preparing polymers containing terminal amines in high purity are described in U.S. patent application Ser. Nos. 11/508,507 and 11/537,172, the contents of each of which are incorporated by reference. For example, polymers having azides react with phosphine-based reducing agent such as triphenylphosphine or an alkali metal borohydride reducing agent such as NaBH₄. Alternatively, polymers including leaving groups react with protected amine salts such as potassium salt of methyl-tert-butyl imidodicarbonate (KNMeBoc) or the potassium salt of di-tert-butyl imidodicarbonate (KNBoc₂) followed by deprotecting the protected amine group. The purity of the polymers containing the terminal amines formed by these processes is greater than about 95% and preferably greater than 99%.

In alternative aspects, polymers having terminal carboxylic acid groups can.be employed in the polymeric delivery systems described herein. Methods of preparing polymers having terminal carboxylic acids in high purity are described in U.S. patent application Ser. No. 11/328,662, the contents of which are incorporated herein by reference. The methods include first preparing a tertiary alkyl ester of a polyalkylene oxide followed by conversion to the carboxylic acid derivative thereof. The first step of the preparation of the PAO carboxylic acids of the process includes forming an intermediate such as t-butyl ester of polyalkylene oxide carboxylic acid. This intermediate is formed by reacting a PAO with a t-butyl haloacetate in the presence of a base such as potassium t-butoxide. Once the t-butyl ester intermediate has been formed, the carboxylic acid derivative of the polyalkylene oxide can be readily provided in purities exceeding 92%, preferably exceeding 97%, more preferably exceeding 99% and most preferably exceeding 99.5% purity.

### C. R₂ and R′₂ Groups

In one aspect of tlie invention, the siRNAs Can be linked to the polymeric portion of the compounds described herein via permanent linkers and releasable linkers whether employed alone or in combination. When the conjugates described herein employ two or more linkers, i.e. (e) (or (e′)) is equal to or greater than 2, the two or more linkers for R₂ (or R′₂) can be the same or different. Regardless of the linker(s) selected, it will be understood that they are attached to the remaining portions of the conjugates using synthetic techniques well known to those of ordinary skill. See also Examples 1-3 below.

In one preferred aspect of the invention, the conjugates described herein contain a siRNA attached to a releasable linker. One advantage of the invention is that the siRNA can be released in a controlled manner.

Among the releasable linkers can be benzyl elimination-based linkers, trialkyl lock-based linkers (or trialkyl lock lactonization based), bicine-based linkers, acid labile linkers, lysosomally cleavable peptides and caplhopsin B cleavable peptides. Among the acid labile linkers can be disulfide bond, hydrazone-containing linkers and thiopropionate-containing linkers. Alternatively, the releasable linkers are intracellular labile linkers, extracellular linkers and acid labile linkers. Preferably, the releasable linkers are intracellular labile linkers and/or acid labile linkers, and the release of siRNA from the conjugates of Formula (I) can be facilitated in cytoplasm.

The releasable linkers have the formula:

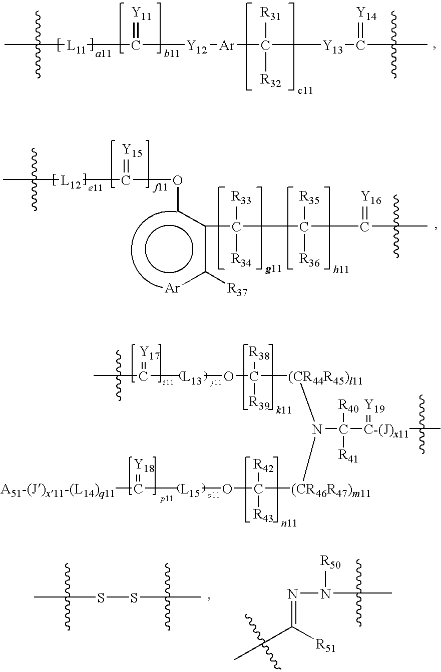

such as —CH═N—NH′—,

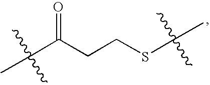

-Val-Cit-,
-Gly-Phe-Leu-Gly-,
-Ala-Leu-Ala-Leu-,
-Phe-Lys-,

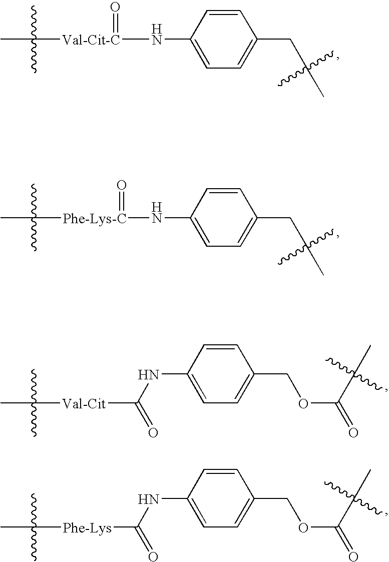

-Val-Cit-C(═O)—CH₂OCH₂—C(═O)—,

-Val-Cit-C(═O)—CH₂SCH₂—C(═O)—, and

—NHCH(CH₃)—C(═O)—NH(CH₂₎₆—C(CH₃)₂—C(═O)—,

wherein

Y₁₁₋₁₉ are independently O, S or NR₄₈;

R₃₁₋₄₈, R₅₀₋₅₁ and A₅₁ are independently selected from among hydrogen, C₁₋₆ alkyls, C₃₋₁₂ branched alkyls, C₃₋₈ cycloalkyls, C₁₋₆ substituted alkyls, C₃₋₈ substituted cyloalkyls, aryls, substituted aryls, aralkyls, C₁₋₆ heteroalkyls, substituted C₁₋₆ heteroalkyls, C₁₋₆ alkoxy, phenoxy and C₁₋₆ heteroalkoxy;

Ar is an aryl or heteroaryl moiety;

L₁₁₋₁₅ are independently selected bifunctional spacers;

J and J′ are independently selected from selected from among moieties actively transported into a target cell, hydrophobic moieties, bifunctional linking moieties and combinations thereof;

(c11), (h11), (k11), (l11), (m11) and (n11) are independently selected positive integers, preferably 1;

(a11), (e11), (g11), (j11), (o11) and (q11) are independently either zero or a positive integer, preferably 1; and

(b11), (x11), (x′11), (f11), (i11) and (p11) are independently zero or one.

The moieties actively transported into a target cell can have the structure of

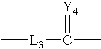

wherein L₃ is a bifunctional linker and Y₄ is O, S or NR₁₁, wherein R₁₁ can be selected from among hydrogen, C₁₋₆ alkyls, C₃₋₁₂ branched alkyls, C₃₋₈ cycloalkyls, C₁₋₆ substituted alkyls, C₃₋₈ substituted cyloalkyls, aryls, substituted aryls, aralkyls, C₁₋₆ heteroalkyls, substituted C₁₋₆ heteroalkyls, C₁₋₆ alkoxy, phenoxy and C₁₋₆ heteroalkoxy.

Various releasable linkers, benzyl elimination based or trialkyl lock based, are described, for example, in commonly assigned U.S. Pat. Nos. 6,180,095, 6,720,306, 5,965,119, 6624,142 and 6,303,569, the contents of each of which are incorporated herein by reference. The bicine-based linkers are also described in commonly assigned U.S. Pat. Nos. 7,122,189 and 7087,229 and U.S. patent application Ser. Nos. 10/557,522, 11/502,108, and 11/011,818, the contents of each of which are incorporated herein by reference.

In some preferred embodiments, the siRNAs are linked to the polymeric portion of the conjugates described herein via acid labile linkers. Without being bound by any theory, the acid labile linkers facilitate release of the oligonucleotides from the parent polymeric compounds within cells and also in lysosome, en do some, or macropinosome.

R₂ and R′₂ can include bifunctional linkers such as amino acids or amino acid derivatives. The amino acids can be among naturally occurring and non-naturally occurring amino acids. Derivatives and analogs of the naturally occurring amino acids, as well as various art-known non-naturally occurring amino acids (D Or L), hydrophobic or non-hydrophobic, are also contemplated to be within the scope of the invention. A suitable non-limiting list of the non-naturally occurring amino acids includes 2-aminoadipic acid, 3-aminoadipic acid, beta-alanine, beta-amino-propionic acid, 2-aminobutyric acid, 4-aminobutyric acid, piperidine acid, 6-aminocaproic acid, 2-aminoheptanoic acid, 2-aminoisobutyric acid, 3-aminoisobutyric acid, 2-aminopimelic acid, 2,4-aminobutyric acid; desmosine, 2,2-diaminopimelic acid, 2,3-diaminopropionic acid, N-ethylglycine, N-ethylasparagine, 3-hydroxyproline, 4-hydroxyproline, isodesmosine, allo-isoleucine, N-methylglycine, sarcosine, N-methylisoleucine, 6-N-methyl-lysine, N-methylvaline, norvaline, norleucine, and ornithine. Some preferred amino acid residues include glycine, alanine, methionine and sarcosine. These bifunctional linkers can be also used for the L₁₁₋₁₅ groups.

Alternatively, R₂ and R′₂ groups can be bifunctional linkers selected from among

—[C(═O)]ᵥ(CR₂₂R₂₃)ₜ[C(═O)]_{v′}—,

—[C(═O)]ᵥ(CR₂₂R₂₃)ₜ—O[C(═O)]_{v′}—,

—[C(═O)]ᵥ(CR₂₂R₂₃)ₜ—NR₂₆[C(═O)]_{v′}—,

—[C(═O)]ᵥO(CR₂₂R₂₃)ₜ[C(═O)]_{v′}—,

—[C(═O)]ᵥO(CR₂₂R₂₃)ₜO[C(═O)]_{v′}—,

—[C(═O)]ᵥO(CR₂₂R₂₃)ₜNR₂₆[C(═O)]_{v′}—,

—[C(═O)]ᵥNR₂₁(CR₂₂R₂₃)ₜ[C(═O)]_{v′}—,

—[C(═O)]ᵥNR₂₁(CR₂₂R₂₃)ₜO[C(═O)]_{v′}—,

—[C(═O)]ᵥNR₂₁(CR₂₂R₂₃)ₜNR₂₆[C(═O)]_{v′}—,

—[C(═O)]ᵥ(CR₂₂R₂₃)ₜO—(CR₂₈R₂₉)_{t′}[C(═O)]_{v′}—,

—[C(═O)]ᵥ(CR₂₂R₂₃)ₜNR₂₆—(CR₂₈R₂₉)_{t′}[C(═O)]_{v′}—,

—[C(═O)]ᵥ(CR₂₂R₂₃)ₜS—(CR₂₈R₂₉)_{t′}[C(═O)]_{v′}—,

—[C(═O)]ᵥO(CR₂₂R₂₃)ₜO—(CR₂₈R₂₉)_{t′}[C(═O)]_{v′}—,

—[C(═O)]ᵥO(CR₂₂R₂₃)ₜNR₂₆—(CR₂₈R₂₉)_{t′}[C(═O)]_{v′}—,

—[C(═O)]ᵥO(CR₂₂R₂₃)ₜS—(CR₂₈R₂₉)_{t′}[C(═O)]_{v′}—,

—[C(═O)]ᵥNR₂₁(CR₂₂R₂₃)ₜO—(CR₂₈R₂₉)_{t′}[C(═O)]_{v′}—,

—[C(═O)]ᵥNR₂₁(CR₂₂R₂₃)ₜNR₂₆—(CR₂₈R₂₉)_{t′}[C(═O)]_{v′}—,

—[C(═O)]ᵥNR₂₁(CR₂₂R₂₃)ₜS—(CR₂₈R₂₉)_{t′}[C(═O)]_{v′}—,

—[C(═O)]ᵥ(CR₂₂R₂₃CR₂₈R₂₉O)ₜNR₂₆[C(═O)]_{v′}—,

—[C(═O)]ᵥ(CR₂₂R₂₃CR₂₈R₂₉O)ₜ[C(═O)]_{v′}—,

—[C(═O)]ᵥO(CR₂₂R₂₃CR₂₈R₂₉O)ₜNR₂₆[C(═O)]_{v′}—,

—[C(═O)]ᵥO(CR₂₂R₂₃CR₂₈R₂₉O)ₜ[C(═O)]_{v′}—,

—[C(═O)]ᵥNR₂₁(CR₂₂R₂₃CR₂₈R₂₉O)ₜNR₂₆[C(═O)]_{v′}—,

—[C(═O)]ᵥNR₂₁(CR₂₂R₂₃CR₂₈R₂₉O)ₜ[C(═O)]_{v′}—,

—[C(═O)]ᵥ(CR₂₂R₂₃CR₂₈R₂₉O)ₜ(CR₂₄R₂₅)_{t′}[C(═O)]_{v′}—,

—[C(═O)]ᵥO(CR₂₂R₂₃CR₂₈R₂₉O)ₜ(CR₂₄R₂₅)_{t′}[C(═O)]_{v′}—,

—[C(═O)]ᵥNR₂₁(CR₂₂R₂₃CR₂₈R₂₉O)ₜ(CR₂₄R₂₅)_{t′}[C(═O)]_{v′}—,

—[C(═O)]ᵥ(CR₂₂R₂₃CR₂₈R₂₉O)ₜ(CR₂₄R₂₅)_{t′}O[C(═O)]_{v′}—,

—[C(═O)]ᵥ(CR₂₂R₂₃)ₜ(CR₂₄R₂₅CR₂₈R₂₉O)_{t′}[C(═O)]_{v′}—,

—[C(═O)]ᵥ(CR₂₂R₂₃)ₜ(CR₂₄R₂₅CR₂₈R₂₉O)_{t′}NR₂₆[C(═O)]_{v′}—,

—[C(═O)]ᵥO(CR₂₂R₂₃CR₂₈R₂₉O)ₜ(CR₂₄R₂₅)_{t′}O[C(═O)]_{v′}—,

—[C(═O)]ᵥO(CR₂₂R₂₃)ₜ(CR₂₄R₂₅CR₂₈R₂₉O)_{t′}[C(═O)]_{v′}—,

—[C(═O)]ᵥO(CR₂₂R₂₃)ₜ(CR₂₄R₂₅CR₂₈R₂₉O)_{t′}NR₂₆[C(═O)]_{v′}—,

—[C(═O)]ᵥNR₂₁(CR₂₂R₂₃CR₂₈R₂₉O)ₜ(CR₂₄R₂₅)_{t′}O[C(═O)]_{v′}—,

—[C(═O)]ᵥNR₂₁(CR₂₂R₂₃)ₜ(CR₂₄R₂₅CR₂₈R₂₉O)_{t′}[C(═O)]_{v′}—,

—[C(═O)]ᵥNR₂₁(CR₂₂R₂₃)ₜ(CR₂₄R₂₅CR₂₈R₂₉O)_{t′}NR₂₆[C(═O)]_{v′}—,

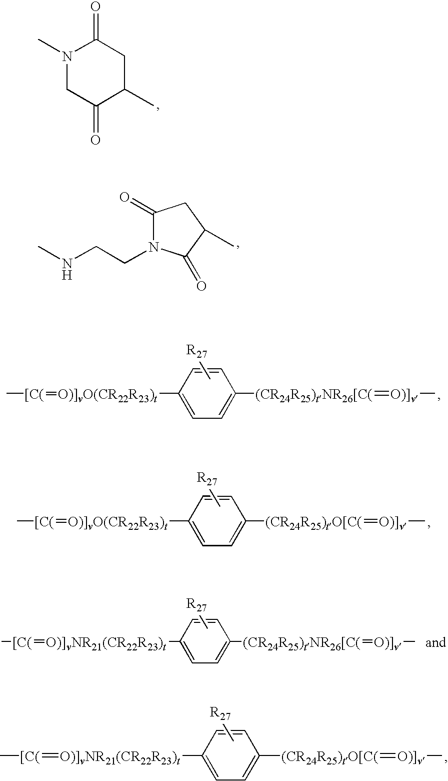

wherein:

R₂₁₋₂₉ are independently selected from among hydrogen, C₁₋₆ alkyls, C₃₋₁₂ branched alkyls, C₃₋₈ cycloalkyls, C₁₋₆ substituted alkyls, C₃₋₈ substituted cyloalkyls, aryls, substituted aryls, aralkyls, C₁₋₆ heteroalkyls, substituted C₁₋₆ heteralkyls, C₁₋₆ alkoxy, phenoxy and C₁₋₆ heteroalkoxy;

(t) and (t′) are independently zero or a positive integer, preferably zero or an integer from about 1 to about 12, more preferably an integer from about 1 to about 8, and most preferably 1 or 2; and

(v) and (v′) are independently zero or 1.

These bifunctional linkers can be also used for the L₁₁₋₁₅ groups.

Preferably, the bifunctional linkers can be selected from among:

—[C(═O)]ᵣNH(CH₂)₂CH═N—NHC(═O)—(CH₂)₂—,

—[C(═O)]ᵣNH(CH₂)₂(CH₂CH₂O)₂(CH₂)₂NH[C(═O)]_{r′}—,

—[C(═O)]ᵣNH(CH₂CH₂)(CH₂CH₂O)₂NH[C(═O)]_{r′}—,

—[C(═O)]ᵣNH(CH₂CH₂)₂NH(CH₂CH₂)_{s′}[C(═O)]_{r′}—,

—[C(═O)]ᵣNH(CH₂CH₂)₂S(CH₂CH₂)_{s′}[C(═O)]_{r′}—,

—[C(═O)]ᵣNH(CH₂CH₂)(CH₂CH₂O)[C(═O)]_{r′}—,

—[C(═O)]ᵣNH(CH₂CH₂)ₛO(CH₂CH₂)_{s′}[C(═O)]_{r′}—,

—[C(═O)]ᵣNH(CH₂CH₂O)(CH₂)NH[C(═O)]_{r′}—,

—[C(═O)]ᵣNH(CH₂CH₂O)₂(CH₂)[C(═O)]_{r′}—,

—[C(═O)]ᵣNH(CH₂CH₂O)ₛ(CH₂)_{s′}[C(═O)]_{r′}—,

—[C(═O)]ᵣNHCH₂CH₂NH[C(═O)]_{r′}—,

—[C(═O)]ᵣNH(CH₂CH₂)₂O[C(═O)]_{r′}—,

—[C(═O)]ᵣNH(CH₂CH₂O)[C(═O)]_{r′}—,

—[C(═O)]ᵣNH(CH₂CH₂)₂[C(═O)]_{r′}—,

—[C(═O)]ᵣNH(CH₂)₃[C(═O)]_{r′}—,

—[C(═O)]ᵣO(CH₂CH₂O)₂(CH₂)[C(═O)]_{r′}—,

—[C(═O)]ᵣO(CH₂)₂NH(CH₂)₂[C(═O)]_{r′}—,

—[C(═O)]ᵣO(CH₂CH₂O)₂NH[C(═O)]_{r′}—,

—[C(═O)]ᵣO(CH₂)₂O(CH₂)₂[C(═O)]_{r′}—,

—[C(═O)]ᵣO(CH₂)₂S(CH₂)₂[C(═O)]_{r′}—,

—[C(═O)]ᵣO(CH₂CH₂)NH[C(═O)]_{r′}—,

—[C(═O)]ᵣO(CH₂CH₂)O[C(═O)]_{r′}—,

—[C(═O)]ᵣO(CH₂)₃NH[C(═O)]_{r′}—,

—[C(═O)]ᵣO(CH₂)₃O[C(═O)]_{r′}—,

—[C(═O)]ᵣO(CH₂)₃[C(═O)]_{r′}—,

—[C(═O)]ᵣCH₂NHCH₂[C(═O)]_{r′}—,

—[C(═O)]ᵣCH₂OCH₂[C(═O)]_{r′}—,

—[C(═O)]ᵣCH₂SCH₂[C(═O)]_{r′}—,

—[C(═O)]ᵣS(CH₂)₃[C(═O)]_{r′}—,

—[C(═O)]ᵣNH(CH₃)₃[C(═O)]_{r′}—,

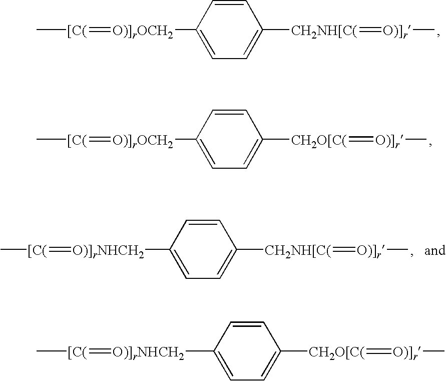

wherein (r) and (r′) are independently zero or 1, provided that both (r) and (r′) are not simultaneously zero.

In yet further alternative aspects of the invention, the bifunctional linkers include:

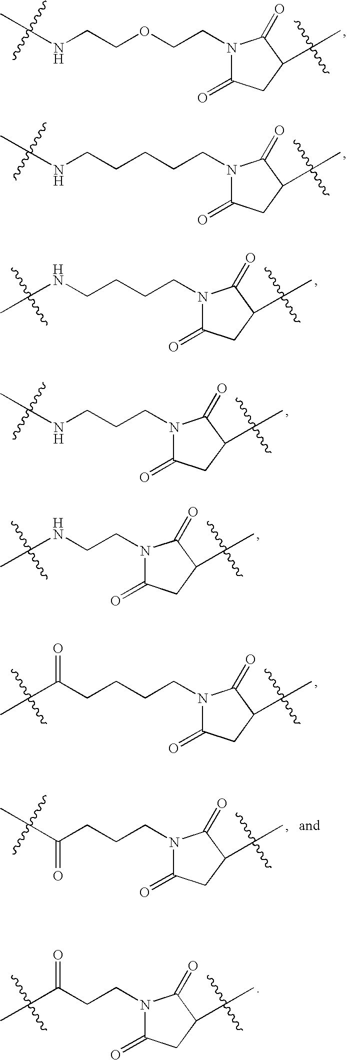

These bifunctional groups allow a second agent to be directly conjugated and therefore eliminate the need of attaching a functional group/for conjugating to a second agent.

In alternative embodiments, the bifunctional linkers include structures corresponding to those shown above and have groups such as vinyl, residues of vinyl sulfone, amino, carboxy, mercapto, thiopropionate, hydrazide, carbamate and the like instead of maleimidyl.

Other permanent or releasable linkers known to those of ordinary skill are also contemplated as being within the conjugates described herein.

D. siRNA-Containing Moieties

The conjugates described herein can be used for delivering various siRNA into cells or tissues.

In order to more fully appreciate the scope of the present invention, the following terms are defined. The artisan will appreciate that the terms, “nucleic acid” or “nucleotide” apply to deoxyribonucleic acid (“DNA”), ribonucleic acid, (“RNA) whether single-stranded or double-stranded, unless otherwise specified, and any chemical modifications thereof. An “oligonucleotide” is generally a relatively short polynucleotide, e.g., ranging in size from about 2 to about 200 nucleotides, or more preferably from about 10 to about 30 nucleotides in length. The oligonucleotides according to the invention are generally synthetic nucleic acids, and are single stranded, unless otherwise specified. The terms, “polynucleotide” and “polynucleic acid” may also be used synonymously herein.

The term “antisense,” or “antisense strand” as used herein, refers to nucleotide sequences which are complementary to a specific DNA or RNA sequence that encodes a gene product or that encodes a control sequence. In the normal operation of cellular metabolism, the sense strand of a DNA molecule is the strand that encodes polypeptides and/or other gene products. Antisense nucleic acid molecules may be produced by any art-known methods, including synthesis by ligating the gene(s) of interest in a reverse orientation to a viral promoter which permits the synthesis of a complementary strand. Once introduced into a cell, this transcribed strand combines with natural sequences produced by the cell to form duplexes. These duplexes then block either the further transcription or translation. The designations “negative” or (−) are also art-known to refer to the antisense strand, and “positive” or (+) are also art-known to refer to the sense strand.

For purposes of the present invention, “complementary” shall be understood to mean that a nucleic acid sequence forms hydrogen bond(s) with another RNA sequence. A percent complementarity, indicates the percentage of contiguous residues in a nucleic acid molecule which can form hydrogen bonds, i.e., Watson-Crick base pairing, with a second nucleic acid sequence, i.e., 5, 6, 7, 8, 9, 10 out of 10 being 50%, 60%, 70%, 80%, 9.0%, and 100% complementary, “Perfectly Complementary” means that all the contiguous residues of a nucleic acid sequence foim hydrogen bonds with the same number of contiguous residues in a second nucleic acid sequence.

The oligonucleotides (analogs) are not limited to a single species of oligonucleotide but, instead, are designed to work with a wide variety of such moieties, it being understood that linkers can attach to one or more of the 3′- or 5′-terminals, usually PO₄ or SO₄ groups of a nucleotide. The nucleic acids molecules contemplated can include a phosphorothioate internucleotide linkage modification, sugar modification, nucleic acid base modification and/or phosphate backbone modification. The oligonucleotides can contain natural phosphorodiester backbone or phosphorothioate backbone or any other modified backbone analogues such as LNA (Locked Nucleic Acid), PNA (nucleic acid with peptide backbone), CpG oligomers, and the like, such as those disclosed at Tides 2002, Oligonucleotide and Peptide Technology Conferences, May 6-8, 2002, Las Vegas, Nev. and Oligonucleotide & Peptide Technologies, 18 & 19 Nov. 2003, Hamburg, Germany, the contents of which are incorporated herein by reference.

Oligonucleotides according to the invention can also optionally include any suitable art-known nucleotide analogs and derivatives, including those listed by Table 1, below.

TABLE 1
Representative Nucleotide Analogs And Derivatives

| | |
|---|---|
| 4-acetylcytidine | 5-methoxyaminomethyl-2-thiouridine |
| 5-(carboxyhydroxymethyl)uridine | beta, D-mannosylqueuosine |
| 2′-O-methylcytidine | 5-methoxycarbonylmethyl-2- |
| | thiouridine |
| 5-carboxymethylaminomethyl-2- | 5-methoxycarbonylmethyluridine |
| thiouridine | |
| 5-carboxymethylamino- | 5-methoxyuridine |
| methyluridine | |
| Dihydrouridine | 2-methylthio-N6- |
| | isopentenyladenosine |
| 2′-O-methylpseudouridine | N-((9-beta-D-ribofuranosyl-2- |
| | methylthiopurine-6- |
| | yl)carbamoyl)threonine |
| D-galactosylqueuosine | N-((9-beta-D-ribofuranosylpurine-6- |
| | yl)N-methylcarbamoyl)threonine |
| 2′-O-methylguanosine | uridine-5-oxyacetic acid-methylester |
| Inosine | uridine-5-oxyacetic acid |
| N6-isopentenyladenosine | Wybutoxosine |
| 1-methyladenosine | Pseudouridine |
| 1-methylpseudouridine | Queuosine |
| 1-methylguanosine | 2-thiocytidine |
| 1-methylinosine | 5-methyl-2-thiouridine |
| 2,2-dimethylguanosine | 2-thiouridine |
| 2-methyladenosine | 4-thiouridine |
| 2-methylguanosine | 5-methyluridine |
| 3-methylcytidine | N-((9-beta-D-ribofuranosylpurine- |
| | 6-yl)-carbamoyl)threonine |
| 5-methylcytidine | 2′-O-methyl-5-methyluridine |
| N6-methyladenosine | 2′-O-methyluridine |
| 7-methylguanosine | Wybutosine |
| 5-methylaminomethyluridine | 3-(3-amino-3-carboxy-propyl)uridine |
| Locked-adenosine | Locked-cytidine |
| Locked-guanosine | Locked-thymine |
| Locked-uridine | Locked-methylcytidine |

Modifications to the oligonucleotides contemplated by the invention include, for example, the addition to or substitution of selected nucleotides with functional groups or moieties that permit covalent linkage of an oligonucleotide to a desirable polymer, and/or the addition or substitution of functional moieties that incorporate additional charge, polarizability, hydrogen bonding, electrostatic interaction, and functionality to an oligonucleotide. Such modifications include, but are not limited to, 2′-position sugar modifications, 5-position pyrimidine modifications, 8-position purine modifications, modifications at exocyclic amines, substitution of 4-thiouridine, substitution of 5-bromo or 5-iodouracil, backbone modifications, methylations, base-pairing combinations such as the isobases isocytidine and isoguanidine, and analogous combinations. Oligonucleotides contemplated within the scope of the present invention can also include 3′ and/of 5′ cap structure See examples of nucleoside analogues described in Freier & Altmanh; Nucl Acid Res., 1997, 25, 4429-4443 and Uhlmann; Curr. Opinion in Drug Development, 2000, 3(2), 293-213, the contents of each of which are incorporated herein by reference.

For purposes of the present invention, “cap structure” shall be understood to mean chemical, modifications, which have been incorporated at either terminus of the oligonucleotide. The cap can be present at the 5′-terminus (5′-cap) or at the 3′-terminus (3′-cap) or can be present on both terminus. A non-limiting examples of the 5′-cap includes inverted abasic residue (moiety); 4′,5′-methylene nucleotide; 1-(beta-D-erythrofuranosyl) nucleotide, 4′-thio nucleotide, carbocyclic nucleotide; 1,5-anhydrohexitol nucleotide; L-nucleotides; alpha-nucleotides; modified base nucleotide; phosphorodithioate linkage; threo-pentofuranosyl nucleotide; acyclic 3′,4′-seco nucleotide; acyclic 3,4-dihydroxybutyl nucleotide; acyclic 3,5-dihydroxypentyl nucleotide, 3′-3′-inverted nucleotide moiety; 3′-3′-inverted abasic moiety; 3′-2′-inverted nucleotide moiety: 3′-2′-inverted abasic moiety; 1,4-butanediol phosphate; 3′-phosphoramidate; hexylphosphate; aminohexyl phosphate; 3′-phosphate; 3′-phosphorothioate; phosphorodithioate; or bridging or non-bridging methylphosphohate moiety. Details are described in WO 97/26270, incorporated by reference herein. The 3′-cap can includes for example 4′,5′-methylene nucleotide; 1-(beta-D-erythrofuranosyl) nucleotide; 4′-thio nucleotide, carbocyclic nucleotide; 5′-amino-alkyl phosphate; 1,3-diamino-2-propyl phosphate, 3-aminopropyl phosphate; 6-aminohexyl phosphate; 1,2-aminododecyl phosphate; hydroxypropyl phosphate: 1,5-anhydrohexitol nucleotide; L-nucleolide; alpha-nucleotide; modified base nucleotide; phosphorodithioate; threo-pentofuranosyl nucleotide; acyclic 3′,4′-seco nucleotide; 3,4-dihydroxybutyl nucleotide; 3,5-dihydroxypentyl nucleotide, 5′-5′-inverted nueleotide moiety; 5′-5′-inverted abasic moiety; 5′-phosphoramidate; 5′-phosphorothioate; 1,4-butanediol phosphate; 5′-amino; bridging and/or non-bridging 5′-phosphoramidate, phosphorothioate and/or phosphorodithioate, bridging or non bridging methylphosphonate and 5′-mercapto moieties. See also Beaucage and Iyer, 1993, Tetrahedron 49, 1925; the contents of which are incorporated by reference herein.

In one preferred aspect of the present invention, the siRNA contemplated is involved in inhibiting or downregulating a gene or protein implicated in the resistance of tumor cells to anticancer therapeutics. For example, any art-known cellular proteins such as BCL2 for downregulation by antisense oligonucleotides, for cancer therapy, can be used for the present invention. Sec U.S. patent application Ser. No. 10/822,205 filed Apr. 9, 2004, the contents of which are incorporated by reference herein. A non-limiting list of preferred therapeutic can include BCL2 siRNA, HIF-1a siRNA and Suryivin siRNA.

In one preferred aspect, each strand of siRNA-containing moiety can include about 18 to about 28 nucleotides in length, more preferably about 20 to about 24 nucleotides, and most preferably about 21 nucleotides complementary to a target gene. The length of the nucleotides can also vary according to the needs of the artisan and the number of the complementary nucleic acids desired. In each strand of siRNA, at least about 14 to 24 nucleotides are preferably perfectly complementary to the nucleotides of the other strand and/or the target gene. siRNA preferably includes about 2 nucleotide-long 3′ overhangs on either end.

In one embodiment, the double stranded siRNA molecule can inhibit or down regulate a gene expression such as BCL2 gene via RNA interference. In one preferred embodiment, the antisense strand of the siRNA molecule contains nucleotide sequence complementary to an RNA of the BCL2 gene for the siRNA molecule to direct cleavage of the RNA via RNA interference.

In more preferred embodiment, the antisense strand of the siRNA-containing moiety includes about 18 to about 28 nucleotides complementary to the nucleic acid sequence of SEQ ID NO: 1, BCL 2 gene is also described in U.S. Pat. Nos. 5,831,066, 6,040,181, 6,414,134 and 6,841,541, the contents of each of which are incorporated by reference herein. One particularly preferred embodiment employs the antisense strand of the siRNA-containing moiety including the nucleic acid sequence of SEQ ID NO: 3.

BCL2 siRNA:

| | |
|---|---|
| SENSE | |
| 5′-GCAUGCGGCCUCUGUUUGAdTdT-3′ | (SEQ ID NO: 2) |
| ANTISENSE | |
| 3′-dTdTCGUACGCCGGAGACAAACU-5′ | (SEQ ID NO: 3) |

where dT represents DNA.

The siRNA molecule employed in the conjugates described herein can be modified with (CH₂)_{w} amino linkers at 5′ or 3′ end of the oligonucleotides, where (w) in this aspect is a positive integer of preferably from about 1 to about 10, preferably 6. The modified oligonucleotides can be NH—(CH₂)_{w}-Oligonucleotide as shown below

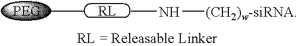

In one embodiment, 5′ end of the sense strand of siRNA is modified. For example, siRNA employed in the polymeric conjugates is modified with a 5-C₆—NH₂.

In alternative aspects, the conjugates described herein can include siRNAs modified with hindered ester-containing (CH₂)_{w} amino linkers. See PCT Application Nos. PCT/USO7/78597 entitled “Hindered Ester-Based Biodegradable Linkers For Oligonucleotide Delivery” and PCT/USO7/78593 entitled “Polyaklylene Oxides Having Hindered Ester-Based Biodegradable Linkers”, the contents of each, of which are incorporated by reference. The polymeric compounds can release the oligonucleotides without amino tail. For example, the oligonucleotides can have the structure:

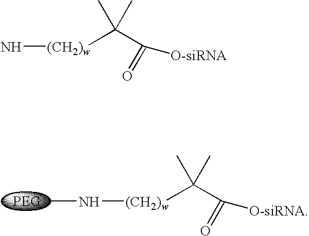

In yet alternative aspects, siRNAs can be modified with (CH₂)_{w} sulfhydryl linkers (thio oligonucleotides). The thio oligonucletides can be used for conjugating directly to cysteine of the linkers or via maleimidyl group. The thio oligonucleotides can have the structure SH—(CH₂)_{w}-Oligonucleotide. The thio oligonucleotides can also include hindered ester having the structure:

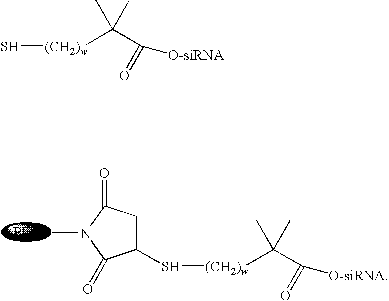

### E. Preferred Embodiments Corresponding to Formula I

For example, the siRNA conjugates prepared in accordance with the present invention are among:

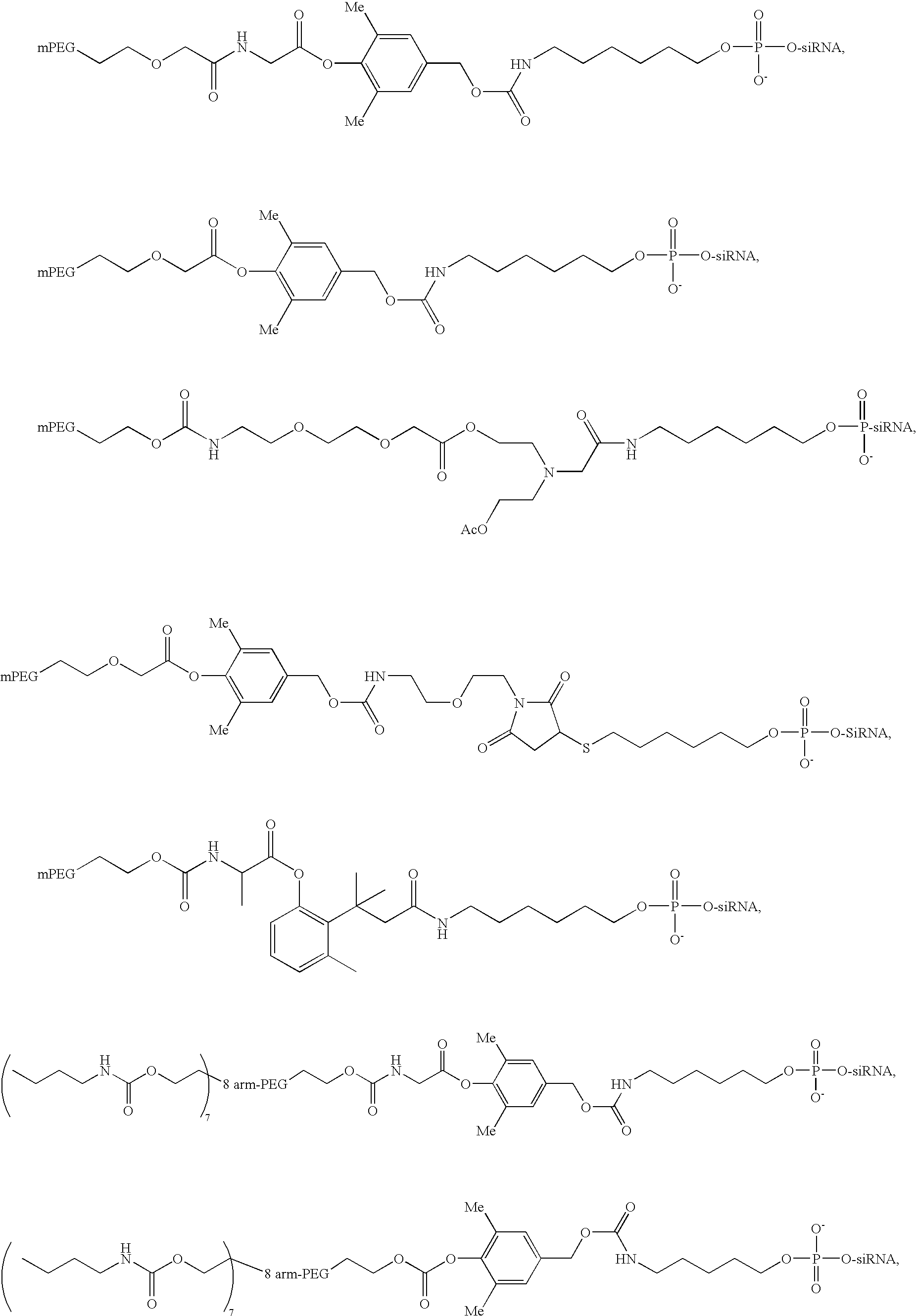

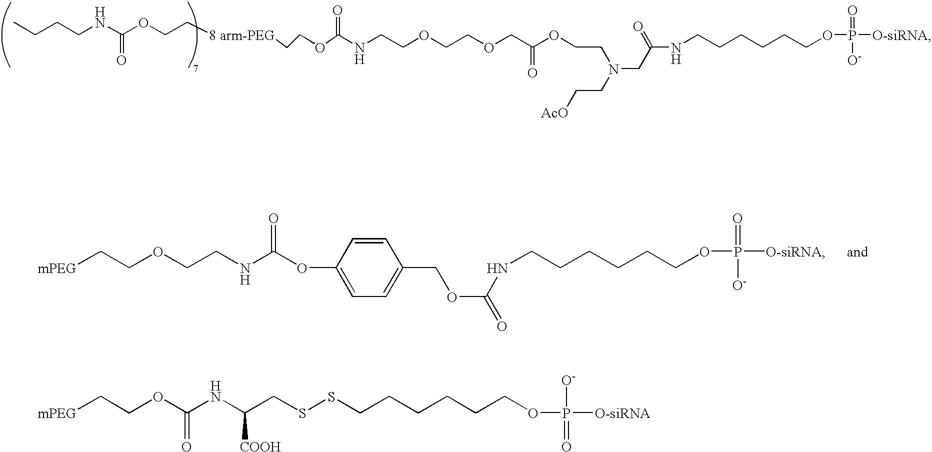

wherein the sense strand of the siRNA-containing moiety is conjugated to the polymer.

In one preferred embodiment, the antisense strand of the siRNA-containing moiety can contain about 18 to about 28 nucleotides complementary to the nucleic acid sequence expressed or overexpressed in cancer cells or tissues. In one particularly preferred embodiment, the siRNA-containing moiety can contain nucleotides complementary to the nucleic acid sequence of SEQ ID NO: 1 such as the nucleic acid sequence of SEQ ID NO: 3.

In one particular embodiment, the conjugates are among:

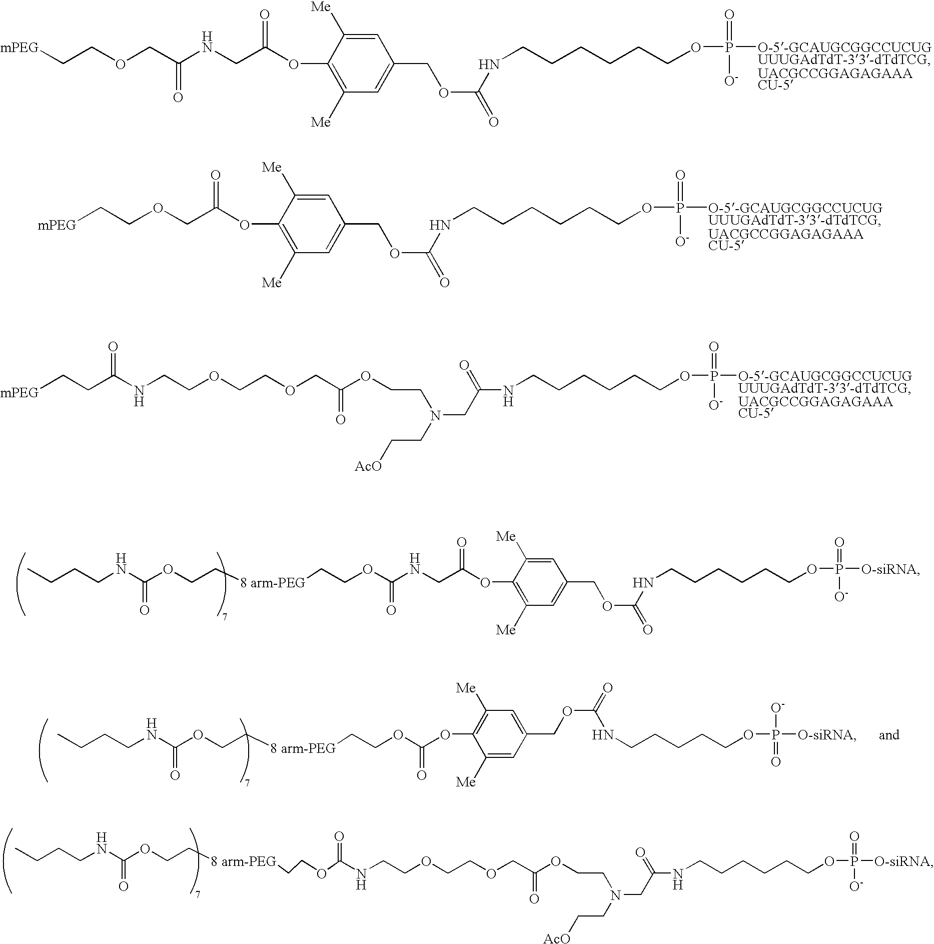

wherein, siRNA has the nucleic acid sequences of SEQ ID NOs: 2 and 3; and the 5′-end of the sense strand of the siRNA is modified to a C6-amino tail for conjugating to PEG linkers.

### F. Synthesis of the Polymeric Delivery Systems

Generally, the siRNA conjugates can be made by first preparing an activated polymer, which in turn reacts with a siRNA containing moiety to provide the polymeric siRNA conjugate. The exact order of addition is not limited to this order and as will be apparent to those of ordinary skill, there are aspects in which the PEG will be first added to the linker followed by the activation of the linker.

In some preferred aspects of the present invention, a polymeric compound containing a OH or a leaving group can first react with a nucleophile containing a releasable linker. The releasable linker is then activated and the activated linker reacts with the functional group of the siRNA containing moiety including a OH, NH₂ or SH group. The polymer containing different activating groups can provide different chemical reactivities toward various nucleophilic moieties. For example, maleimidyl group and vinylsulfone groups can react selectively with SH containing moieties. Details concerning some preferred aspects of this embodiment are provided in the Examples below.

All reactions described herein employ standard chemical reactions with necessary steps and conditions known to those of ordinary skill. The synthetic reactions described herein therefore do not require undue experimentation.

The attachment of the linker moieties to PEG or other polymer can be done using standard chemical synthetic techniques well known to those of ordinary skill using the polymer and coupling reagent or by utilizing the activated polymers. The activated polymer portion such as SC-PEG, PEG-amine, PEG acids, etc. can be obtained from either commercial sources or synthesized by the artisan without undue experimentation.

Attachment of linker moieties to the polymer portion is carried out in the presence of a coupling agent. A non-limiting list of suitable coupling agents include 1,3-diisopropylcarbodiimide (DIPC), any suitable dialkyl carbodiimides, 2-halo-1-alkyl-pyridinium halides (Mukaiyama reagents), 1-(3-dimethylaminopropyl)-3-ethyl carbodiimide (EDC), propane phosphonic acid cyclic anhydride (PPACA) and phenyl dichlorophosphates, etc. which are available, for example from commercial sources such as Sigma-Aldrich Chemical, or synthesized using known techniques.

Preferably, the reactions are earned out in an inert solvent such as methylene chloride, chloroform, DMF or mixtures thereof. The reactions can be preferably conducted in the presence of a base, such as dimethylaminopyridine (DMAP), diisopropylethylamine, pyridine, triethylamine, etc. to neutralize any acids generated. The reactions can be carried out at a temperature from about 0° C. up to about 22° C. (room temperature).

In one aspect, the polymeric compounds reacting with siRNA containing moieties can be prepared in aqueous solvent at pH of from about 5 to about 10, preferably neutral pH using buffer solution such as PBS and room temperature to conserve the integrity of siRNA duplex structure.

Preferably, the sense strand of the siRNA containing moieties can be one of following types:

(i) an oligonucleotide modified with a (CH₂)_{w} amino linker at 5-′ or 3′-end of the oligonucleotide;

(ii) an oligonucleotide modified with a (CH₂)_{w} sulfhydryl linker at 5-′ or 3′-end of the oligonucleotide;

(iii) an oligonucleotide modified with a (CH₂)_{w} amino linker or (CH₂)_{w} sulfhydryl linker containing a hindered ester. (w) in this aspect is a positive integer of preferably from about 1 to about 10, preferably 6.

The modification of the sense strand of the siRNA can be achieved by standard techniques known in the art. Some C₆—NH₂ modified sense strand are also commercially available from TriLink BioTechnologies in San Diego, Calif. The modified sense strand and the unmodified antisense strand then anneal and form the duplex structure of siRNA. The inventive selective conjugation of the sense strand of the siRNA duplex to the activated polymer can be obtained in part because aromatic amines in the modified siRNA duplex are not nucleophilic enough to react under the conjugation reaction conditions such as aqueous condition and pH 5-10. The reaction thus proceeds at the terminal containing the more nucleophilic modification on the sense strand.

Description concerning the formation of hindered ester-containing oligonucleotides is described in commonly-assigned PCT Application Nos. PCT/US07/78597 and PCT/US07/78593, the contents of which are incorporated herein by reference.

In yet another aspect, the siRNA conjugates prepared in accordance with the present invention can contain one or more releasable linkers. See PCT Patent Application No. PCT/USO7/78598, the contents of which are incorporated herein by reference.

### G. Methods of Treatment

In view of the above, there are also provided methods of down-regulating or inhibiting a gene expression in human cells or tissues. The downregulation or inhibition of gene expression can be achieved in vivo and/or in vitro. The methods include contacting human cells or tissues with siRNA conjugates of Formula (I) described herein. Once the contacting has occurred, successful inhibition or down-regulation of gene expression such as in mRNA or protein levels shall be deemed to occur when at least about 10%, preferably at least about 20% or higher is realized when measured in vivo or in vitro.

For purposes of the present invention, “inhibiting” or “down-regulating” shall be understood to mean that the expression of the gene, or level of RNAs or equivalent RNAs encoding one or more protein subunits, or activity of one or more protein subunits, such as BCL2, is reduced below that observed in the absence of the conjugates described herein.

In one preferred aspect of the invention, the present invention is directed to siRNA conjugates, which are targeted to a gene associated and overexpressed in cancer cells or tissues such as a gene encoding BCL2. In one particular embodiment, the antisense strand of the siRNA molecules contains about 18 to about 28 nucleotides complementary to the nucleic acid sequence of SEQ ID NO: 1.

The cancer cells or tissues can be from one or more of the following: solid tumors, lymphomas, small cell lung cancer, acute lymphocytic leukemia (ALL), pancreatic cancer, glioblastoma, ovarian cancer, gastric cancer, breast cancer, colorectal cancer, prostate cancer, ovarian cancer and brain tumors, etc.

In another aspect, there are also provided methods of treating a patient having a cancer; treating neoplastic disease, reducing tumor burden, preventing metastasis of neoplasms or preventing recurrences of tumor/neoplastic growths in mammals. The methods include administering an effective amount of a pharmaceutical composition containing the siRNA conjugates of Formula (I) to a patient in need thereof. For example, if an unconjugated siRNA (for example, native BCL2 siRNA) has efficacy against certain cancer or neoplastic cells, the method would include delivering a polymer conjugate containing the siRNA to the cells having susceptibility to the native siRNA. The delivery can be made in vivo as part of a suitable pharmaceutical composition or directly to the cells in an ex vivo environment. In one particular treatment, the polymeric conjugates including siRNA molecule (SEQ ID NOs: 2 and 3) can be used.

In yet another aspect, the present invention provides methods of inhibiting the growth or proliferation of cancer cells in vivo or in vitro. The methods include contacting cancer cells with the siRNA conjugates described herein. Preferably, the present invention provides methods of inhibiting the growth of lymphoma or leukemia cells in vivo or in vitro wherein the cells express BCL2 gene. Lymphoma or leukemia cells contact the siRNA released from the conjugates described herein. The antisense strand complementary to siRNA expressed from human BCL2 gene inhibits growth of the lymphoma or leukemia cells and reduce expression of the BCL2 gene in the lymphoma or leukemia cells. Alternatively, the present invention provides methods of modulating apoptosis in cancer cells.

In yet anther aspect, there are also provided methods of increasing the sensitivity of cancer cells or tissues to chemotherapeutic agents in vivo or in vitro. In one particular aspect, the methods include introducing the siRNA conjugates described herein to cancer cells to reduce BCL2 expression in the cancer cells or tissues, wherein the siRNA binds to mRNA expressed from the BCL2 gene and reduces BCL2 gene expression.

In yet another aspect, there are provided methods of killing tumor cells in vivo or in vitro. The methods include introducing siRNA conjugates described herein to tumor cells to reduce gene expression such as BCL2 gene and contacting the tumor cells with an amount of at least one chemotherapeutic agent sufficient to kill a portion of the tumor cells. Thus, the portion of tumor cells killed can be greater than the portion which would have been killed by the same amount of the chemotherapeutic agent in the absence of the siRNA conjugates described herein.

In a further aspect of the invention, a chemotherapeutic agent can be used in combination, simultaneously or sequentially, with the methods employing the siRNA conjugates described herein. The siRNA conjugates described herein can be administered concurrently with the chemotherapeutic agent or after the administration of the chemotherapeutic agent. Thus, the siRNA conjugates can be administered during or after treatment of the chemotherapeutic agent.

For example, a non-liming list of the chemotherapeutic agents includes;

(i) DNA topoisomerase inhibitor: adriamycin, amsacrine, camptothecin, CPT-11, SN38, daunorubicin, dactinomycin, doxorubicin, eniposide, epirubicin, etoposide, idarubicin, or mitoxantrone;

(ii) microtubule inhibiting drug, such as a taxane, including paclitaxel, docetaxel, vincristin, vinblastin, nocodazole, epothilones and navelbine;

(iii) DNA damaging agent: actinomycin, amsacrine, anthracyclines, bleomycin, busulfan, camptothecin, carboplatin, chlorambucil, cisplatin, cyclophosphamide, cytoxan, dactinomycin, daunorubicin, docetaxel, doxorubicin, epirubicin, hexamethylmelamineoxaliplatin, iphosphamide, melphalan, merchlorehtamine, mitomycin, mitoxantrone, nitrosourea, plicamycin, procarbazine, taxol, taxotere, teniposide, triethylenethiophosphoramide or etoposide (VP16);

(iv) antimetabolite: folate antagonist; and

(v) nucleoside analog: 5-fluorouracil; cytosine arabinoside, azacitidine, 6-mercaptopurine, azathioprine; 5-iodo-2′-deoxyuridine; 6-thioguanine, 2-deoxycoformycin. cladribine, cytarabine, fludarabine, mercaptopurine, thioguanine, pentostatin, AZT (zidovudine), ACV, valacylovir, famiciclovir, acyclovir, cidofovir, penciclovir, ganciclovir, Ribavirin, ddC, ddI (zalcitabine), lamuvidine, Abacavir, Adefovir, Didanosine, d4T (stavudine), 3TC, BW 1592, PMEA/bis-POM PMEA, ddT, HPMPC, HPMPG, HPMPA, PMEA, PMEG, dOTC; DAPD, Ara-AC, pentostatin, dihydro-5-azacytidine, tiazofurin, sangivamycin, Ara-A (vidarabine), 6-MMPR, 5-FUDR (floxuridine), cytarabine (Ara-C; cytosine arabinoside), 5-azacytidine (azacitidine), HBG [9-(4-hydroxybutyl)guanine], (1S,4R)-4-[2-amino-6-cyclopropyl-amino)-9H-purin-9-yl]-2-cyclopentene-1-m-ethanol succinate (“159U89”), uridine, thymidine, idoxuridine, 3-deazauridine, cyclocytidine, dihydro-5-azacytidine, triciribine, ribavirin, fludrabine, Acyclovir, 1-beta-D-arabinofuranosyl-E-5-(2-bromovinyl)uracil, 2-fluorocarbocyclic-2′-deoxyguanosine; 6′-fluorocarbocyclic-2′-deoxyguanosine; 1-(beta-D-arabinofuranosyl)-5(E)-(2-iodovinyl)uracil; {(1r-1alpha,2 beta,3 alpha)-2-amino-9-(2,3-bis(hydroxymethyl)cyclobut-yl)-6H-purin-6-one}Lobucavir, 9H-purin-2-amine, 9-((2-(1-methylethoxy)-1-((1-methylethoxy)methyl)ethoxy)methyl)-(9Cl); trifluorothymidine, 9→(1,3-dihydroxy-2-propoxy)methylguanine (ganciclovir), 5-ethyl-2′-deoxyuridine; E-5-(2-bromovinyl)-2′-deoxyuridine; 5-(2-chloroethyl)-2′-deoxyuridine, buciclovir, 6-deoxyacyclovir; 9-(4-hydroxy-3-hydroxymethylbut-1-yl)guanine, E-5-(2-iodovinyl)-2′-deoxyuridine, 5-vinyl-1-β-D-arabinofuranosyluracil, 1-β-D-arabinofuranosylthymine; 2′-nor-2′deoxyguanosine; and 1-β-D-arabinofuranosyladenine.

Other potential anti-cancer agents are selected from altretamine, aminoglutethimide, amsacrine, anastrozole, asparaginase, bcg, bicalutamide, bleomycin, buserelin, busulfan, calcium folinate, campothecin, capecitabine, carboplatin, carinustine, chlorambucil, cisplatin, cladribine, clodronate, colchicine, crisantaspase, cyclophosphamide, cyproterone, cytarabine, dacarbazine, dactinomycin, daunorubicin, dienestrol, diediylstilbestrol, docetaxel, doxorubicin, epirubicin, estradiol, estramustine, etoposide, exemestane, filgrastim, fludarabine, fludrocortisone, fluorouracil, fluoxymesterone, flutamide, gemcitabine, genistein, goserelin, hydroxyurea, idarubicin, ifosfamide, imatinib, interferon, irinotecan, ironotecan, letrozole, leucdvorin, leuprolide, levamisole, lomustine, mechlorethamine, medroxyprogesterone, megestrol, melphalan, mercaptopurine, mesna, methotrexate, mitomycin, mitotane, mitoxantrone, nilutamide, nocodazole, octreotide, oxaliplatin, paclitaxel, pamidronate, pentostatin, plicamycin, porfimer, procarbazine, raltitrexed, rituximab, streptozoein, suramin, tamoxifen, temozolomide, teniposide, testosterone, thioguanine, thiotepa, titandcene dichloride, topotecan, trastuzumab, tretinoin, vinblastine, vincristine, vindesine, and vinorelbine. Other numerous anti-cancer agents are listed in US Patent Publication No. 2006/0135468, the contents of which are incorporated herein by reference.

In alternative aspects, the present invention provides methods of treatment for various medical conditions in mammals. The pharmaceutical composition includes the siRNA conjugates described herein can be used for treatment of many different diseases. Briefly stated, any siRNA which can be attached to the PEG polymer can be administered to cells in vivo or in vitro in need of such treatment. Any siRNA which has therapeutic effects in the unconjugated state can be introduced into cells in its conjugated form, made as described herein.

### H. Compositions/Formulations

Pharmaceutical compositions including the siRNA conjugates of the present invention may be formulated in conjunction with one or more physiologically acceptable carriers comprising excipients and auxiliaries which facilitate processing of the active compounds into preparations which can be used pharmaceutically. Proper formulation is dependent upon the route of administration chosen, i.e. whether local or systemic treatment is treated. Parenteral routes are preferred in many aspects of the invention.

Administration of pharmaceutical compositions, containing the siRNA conjugates described herein may be oral, pulmonary, topical including epidermal, transdermal, ophthalmic and to mucous membranes including vaginal and rectal delivery or parenteral including intravenous, intraarterial, subcutaneous, intraperitoneal or intramuscular injection or infusion. In one embodiment, the siRNA conjugate is administered TV, IP or as a bolus injection.

For injection, including, without limitation, intravenous, intramuscular and subcutaneous injection, the compounds of the invention may be formulated in aqueous solutions, preferably in physiologically compatible buffers such as physiological saline buffer or polar solvents including, without limitation, a pyrrolidone or dimethylsulfoxide.

The compounds may also be formulated for parenteral administration, e.g., by bolus injection or continuous infusion. Formulations for injection may be presented in unit dosage form, e.g., in ampoules or in multi-dose containers. Useful compositions include without limitation, suspensions, solutions or emulsions in oily or aqueous vehicles, and may contain adjuncts such as suspending, stabilizing and/or dispersing agents. Pharmaceutical compositions for parenteral administration include aqueous solutions of a water soluble form, such as, without limitation, a salt (preferred) of the active compound. Additionally, suspensions of the active compounds may be prepared in a lipophilic vehicle. Suitable lipophilic vehicles include fatty oils such as sesame oil, synthetic fatty acid esters such as ethyl oleate and triglycerides, or materials such as liposomes. Aqueous injection suspensions may contain substances that increase the viscosity of the suspension, such as sodium carboxymethyl cellulose, sorbitol, or dextran. Optionally, the suspension may also contain suitable stabilizers and/or agents that increase the solubility of the compounds to allow for the preparation of highly concentrated solutions. Alternatively, the active ingredient may be in powder form for constitution with a suitable vehicle, e.g., sterile, pyrogen-free water, before use.

For oral administration, the compounds can be formulated by combining the active compounds with pharmaceutically acceptable carriers well-known in the art. Such carriers enable the compounds of the invention to be formulated as tablets, pills, lozenges, dragees, capsules, liquids, gels, syrups, pastes, slurries, solutions, suspensions, concentrated solutions and suspensions for diluting in the drinking water of a patient, premixes for dilution in the feed of a patient, and the like, for oral ingestion by a patient. Pharmaceutical preparations for oral use can be made using a solid excipient, optionally grinding the resulting mixture, and processing the mixture of granules, after adding other suitable auxiliaries if desired, to obtain tablets or dragee cores. Useful excipients are, in particular, fillers such as sugars, including lactose, sucrose, maimitol, or sorbitol, cellulose preparations such as, for example, maize starch, wheat starch, rice starch, and potato starch and other materials such as gelatin, gum tragacanth, methyl cellulose, hydroxypropyl-methylcellulose, sodium carboxy-methyl cellulose, and/or polyvinylpyrrolidone (PVP). If desired, disintegrating agents may be added, such as cross-linked polyvinyl pyrrolidone, agar, or alginic acid. A salt such as sodium alginate may also be used.

For administration by inhalation, the compounds of the present invention can conveniently be delivered in the form of an aerosol spray using a pressurized pack or a nebulizer and a suitable propellant.

The compounds may also be formulated in rectal compositions such as suppositories or retention enemas, using, e.g., conventional suppository bases such as cocoa butter or other glycerides.

In addition to the formulations described previously, the compounds may also be formulated as depot preparations. Such long acting formulations may be administered by implantation (for example, subcutaneously or intramuscularly) or by intramuscular injection. A compound of this invention may be formulated for this route of administration with suitable polymeric or hydrophobic materials (for instance, in an emulsion with a pharmacologically acceptable oil), with ion exchange resins, or as a sparingly soluble derivative such as, without limitation, a sparingly soluble salt.

Other delivery systems such as liposomes and emulsions can also be used.

Additionally, the conjugates may be delivered using a sustained-release system, such as semi-permeable matrices of solid hydrophobic polymers containing the therapeutic agent. Various sustained-release materials have been established and are well known by those skilled in the art.

### I. Dosages

Determination of a therapeutically effective amount is well within the capability of those skilled in the art, especially in light of the disclosure herein.

For any conjugate used in the methods of the invention, the therapeutically effective amount can be estimated initially from in vitro assays. Then, the dosage can be formulated for use in animal models so as to achieve a circulating concentration range that includes the effective dosage. Such information can then be used to more accurately determine dosages useful in patients.

The amount of the composition, e.g., used as a prodrug, that is administered will depend upon the parent molecule included therein (i.e., efficacy of an unconjugated siRNA). Generally, the amount of prodrug used in the treatment methods is that amount which effectively achieves the desired therapeutic result in mammals. Naturally, the dosages of the various prodrug compounds will vary somewhat depending upon the parent compound (siRNA), rate of in vivo hydrolysis, molecular weight of the polymer, etc. In addition, the dosage, of course, can vary depending upon the dosage form and route of administration. In general, however, the siRNA conjugates described herein can be administered in amounts ranging from about 1 mg/kg/week to about 1 g/kg/week, preferably from about 1 to about 500 mg/kg/week and more preferably from 1 to about 100 mg/kg/week (i.e., from about 2 to about 60 mg/kg/week). The range set forth above is illustrative and those skilled in the art will determine the optimal dosing of the prodrug selected based on clinical experience and the treatment indication. Moreover, the exact formulation, route of administration and dosage can be selected by the individual physician in view of the patient's condition. Additionally, toxicity and therapeutic efficacy of the compounds described herein can be determined by standard pharmaceutical procedures in cell cultures or experimental animals using methods well-known in the art.

In more preferred aspects, the treatment of the present invention includes administering the siRNA conjugates described herein in an amount of from about 2 to about 50 mg/kg/dose, and preferably from about 5 to about 30 mg/kg/dose to a mammal.

Alternatively, the delivery of the siRNA conjugates described herein includes contacting a concentration of siRNA of from about 0.1 to about 1000 nM, preferably from about 10 to about 1000 nM with tumor cells or tissues in vivo or in vitro.

The compositions may be administered once daily or divided into multiple doses which can be given as part of a multi-week treatment protocol. The precise dose will depend on the stage and severity of the condition, the susceptibility of the tumor to the polymer-prodrug composition, and the individual characteristics of the patient being treated, as will be appreciated by one of ordinary skill in the art.

In all aspects of the invention where polymeric conjugates are administered, the dosage amount mentioned is based on the amount of siRNA molecule rather than the amount of polymeric conjugate administered. It is contemplated that the treatment will be given for one or more days until the desired clinical result is obtained. The exact amount, frequency and period of administration of the compound of the present invention will vary, of course, depending upon the sex, age and medical condition of the patent as well as the severity of the disease as determined by the attending clinician.

Still further aspects include combining the compound of the present invention described herein with other anticancer therapies for synergistic or additive benefit.

### Examples

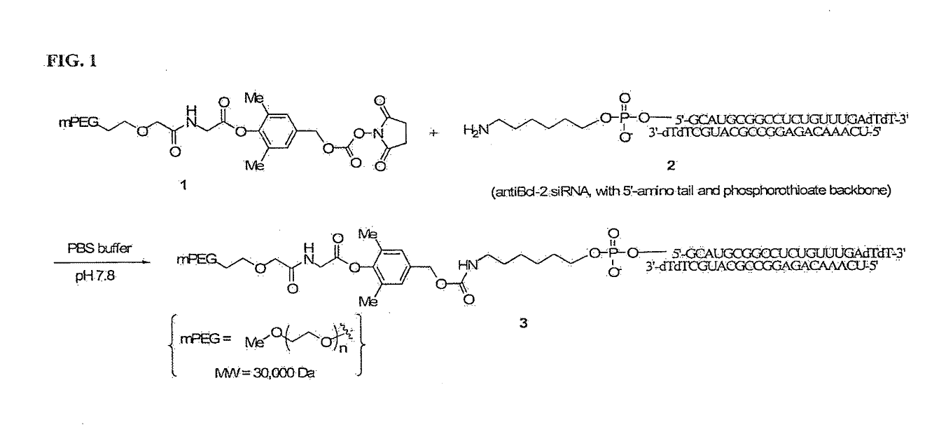

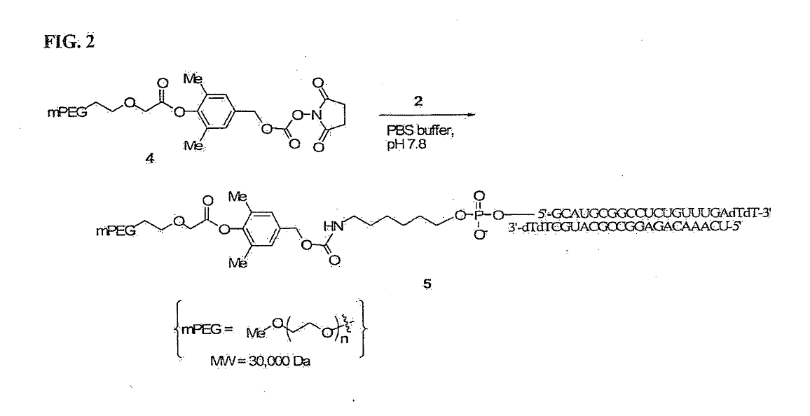

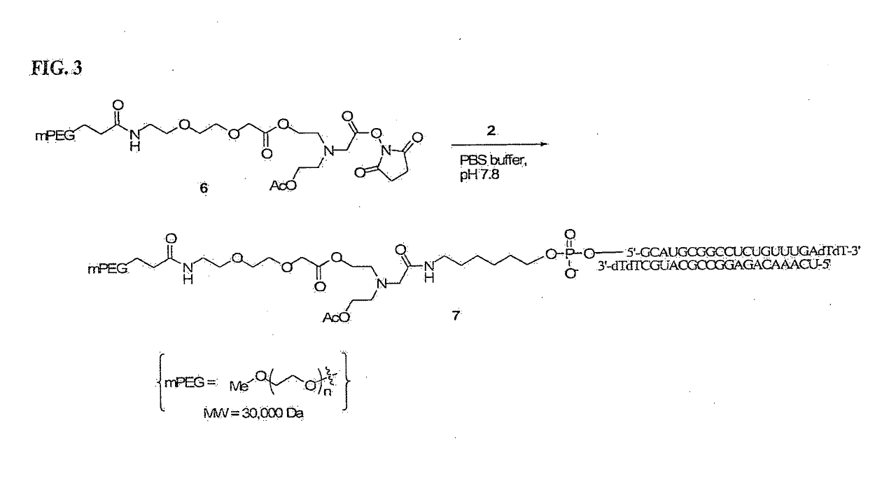

The following examples serve to provide further appreciation of the invention but are not meant in any way to restrict the scope of the invention. The bold-faced numbers recited in the Examples correspond to those shown in FIGS. 1-3.
General Procedures. All the conjugation reactions between PEG linkers and siRNA were carried out in PBS buffer systems at room temperature. Anion-exchange chromatography was used to separate the PEG-siRNA conjugates from non-reacted excess PEG linkers and native siRNA to give pure products.
HPLC Method. The reaction mixtures and the purity of intermediates and final products were monitored by a Beckman Coulter System Gold® HPLC instrument. It employs a Phehomenex Jupiter® 300A C18 reversed phase column (150×4.6 mm) with a photodiode array equipped UV detector, using a gradient of 25-35 % acetohitrile in 50 mM TEAA buffer at a flow rate of 1 mL/min. The anion exchange chromatography was run on AKTA explorer 100A from GE healthcare (Amersham Biosciences) using Poros 50HQ strong anion exchange resin from Applied Biosystems packed in an AP-Empty glass column from Waters. Desalting was achieved by using HiPrep 26/10 desalting columns from Amersham Biosciences.

### Example 1

### Preparation of Compound 3

A solution of compound 1 (330 mg, 0.011 mmol, 30 eq) and compound 2 (5 mg, 0.37 μmol, 1.0 eq) in PBS buffer (2.5 ml, pH 7.4) was stirred at room temperature for 5 hours. The reaction was diluted in Milli-Q water (25 mL) and loaded on a HQ/10 Poros strong anion exchange column (10 mm×60 mm, bed volume ˜6 mL). The un-reacted PEG linkers were removed. The fractions containing pure product were pooled and lyophilized to yield pure compound 3 (6.5 mg, 0.15 μmol, 40%).

### Example 2

### Preparation of Compound 5

Compound 4 was subjected to the conditions described in Example 1 to provide compound 5.

### Example 3

### Preparation of Compound 7

Compound 6 was subjected to the conditions described in Example 1 to provide compound 7.

### Example 4

### In Vitro Stability Studies in Saline

The stability of PEGylated siRNA was measured to determine the effect of conjugation of siRNA to PEG polymer. The native siRNA (SEQ ID NOs: 2 and 3) or PEGylated siRNA conjugates (compounds 3, 5 and 7) were dissolved in saline for about 0.7 mg/mL concentration. An aliquot of 100 μL of each of the test compounds was added to art individual Eppendorf tube, which was incubated at 37° C. for 30 minutes, 1 hour, 2 hours, 4 hours, 6 hours, and 24 hours respectively. The samples at each time point were analyzed by injecting 50-200 μL of aliquot into HPLC followed by analyzing the area of the peak corresponding to the PEGylated siRNA. The assay was repeated in duplicate for each of the test compounds. The results are set forth in Table 1 below.

### Example 5

### In Vitro Stability Studies in Rat and Human Plasma

Kinetics studies of native siRNA and PEGylated siRNAs in human and rat plasmas were conducted to evaluate their in vitro T_{1/2}. The native siRNA or PEGylated siRNA conjugates (compounds 3, 5 and 7) were dissolved in fresh plasma for ˜0.7 mg/mL concentration. An aliquot of 100 μL of each of test compounds was added to each individual Eppendorf tube, which was incubated at 37° C. for 5 minutes, 15 minutes, 30 minutes, 1 hour, 2 hours, 4 hours, 6 hours, and 24 hours, respectively. The native siRNA was extracted with phenol-chloroform and the PEG-siRNA conjugates were extracted with organic solvent acetonitrile/methanol before injecting to HPLC. Assay was repeated in duplicate for each test compound.

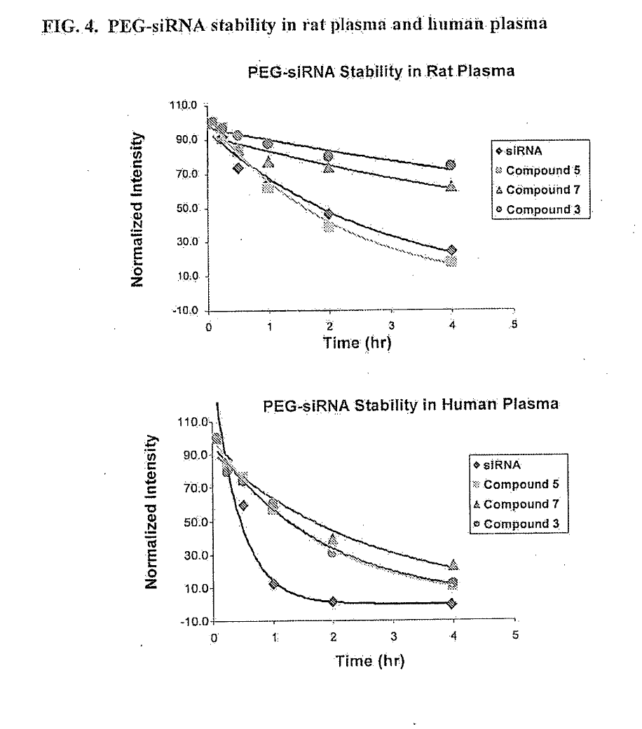

The in vitro stability properties of the PEG-siRNA conjugates are set forth in Table 1 and the stability curves are also shown in FIG. 4.

TABLE 1
In vitro properties of PEG-siRNA conjugates

| | | Stability (24 h, | | | |
|---|---|---|---|---|---|
| | | saline, | | T_{1/2} (hour, | % (W/W) |
| | | % native siRNA | T_{1/2} (hour, | Human | Active in |
| Compound | MW | released) | Rat Plasma) | Plasma) | each conjugate |
| Native-siRNA | 13509 | Stable | 1.80 | 0.29 | 100 |
| Compound 3 | 43771 | <1% | 1.54 | 1.46 | 31 |
| Compound 5 | 43714 | <1% | 1.54 | 1.34 | 31 |
| Compound 7 | 43720 | 10% | 7.7 | 2.03 | 31 |

The results indicate that the PEG-siRNA conjugates have enhanced stability towards nucleases in plasma.

### Example 6

### In Vitro Efficacy Study Using Western Blot

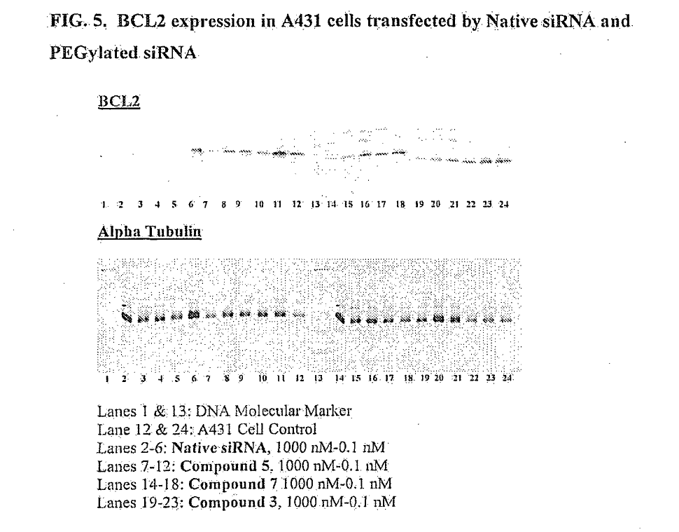

In vitro efficacy studies for siRNA and three PEG-siRNA conjugates (compounds 3, 5 and 7) and native siRNA were performed by using western blot. 0.1 nM to 1,000 nM of each of the test compounds was mixed, with SiLenFect (BioRad) and added onto 10,000 of AK431K5 cells in PRMI1640 medium and incubated at 37° C. at the atmosphere of 5% CO₂ for 72 hours. After incubation, AK431K5 cell proteins were extracted from AK431K5 cells transfected with each test compound, separated on 4-20% SDS-PAGE and transferred onto Hybond membrane. The membrane was incubated in 1/1000 dilution of monoclonal antibody to BCL2 (Santa-Cruz Biotechnology SC7328) and monoclonal antibody to Alpha-tubulin (Santa-Cruz Biotechnology SC5286) at 4° C. overnight, then incubated in 1/2,000 dilution of anti-mouse IgG (Sant-Cruz,SC 2031) at room temperature for 1 hour. The membrane was developed in chemiluminescent reagents. The results from western blot are shown in FIG. 5. The results show that the native siRNA and PEG-siRNA conjugates exhibited a dose-dependent down-regulation of BCL2 protein expression. This property can be advantages in treatment of cancers because clinicians adjust dosage of therapeutic oligonucleotides depending on the heed of patients.

### Example 7

### In Vitro Efficacy Study Using RT-PCR

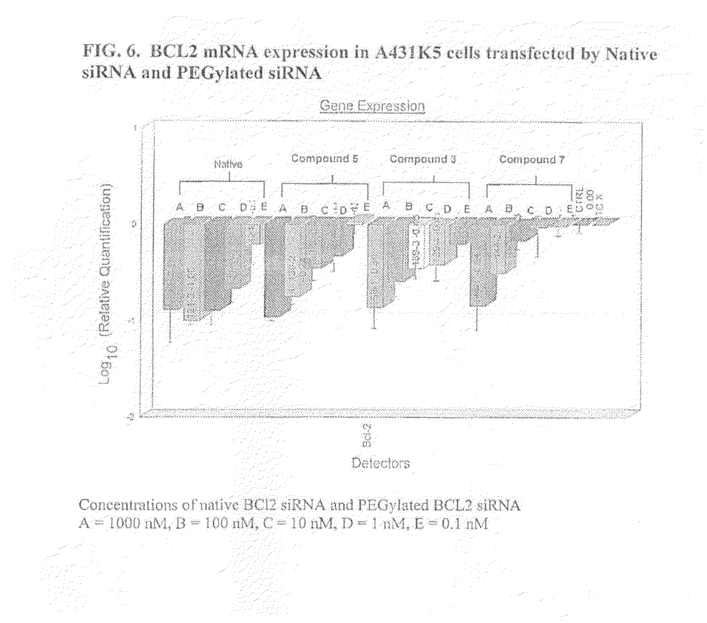

In vitro efficacy studies for three PEG-siRNA conjugates (compounds 3, 5 and 7) and native siRNA were performed by using RT-PCR. The total RNA was extracted from H460 cells transfected with each of the test compounds, following instructions of RNAqueous lot (AmBion). 100 ng of total RNA from each sample was analyzed by relative quantification assay using TagMan gene expression assay (AppliedBiosystems Hs00608023 m1). 18S rRNA gene was used as endogenous control. The results from RT-PCR are shown in FIG. 6. The results show that native siRNA and PEG-siRNA conjugates exhibited a dose-dependent down-regulation of BCL2 mRNA expression. The results indicate that the siRNA delivery technology according to the present invention allows cellular uptake and therapeutic use of siRNA.

### Example 8

### Pharmacokinetics of PEG-siRNA Conjugates in ICR Mice

Thirty (30) female mice per test compound group were administered a single slow-bolus intravenous injection via the lateral tail vein at 50 mg/kg siRNA or siRNA equivalent for each PEG-siRNA conjugate (compounds 3, 5 and 7). Following administration the mice (3/group) were bled by cardiac puncture into EDTA containing tubes at the time points given below. The plasma was collected following centrifugation of the blood for 5 min at 400×g and immediately frozen at −80° C. on dry ice. Five (5) untreated mice were bled as negative controls. The concentration of free siRNA and PEG-siRNA in the plasma was determined by HPLC. Pharmacokinetic parameters were determined using the WinNonlin software, Pharsight.

Each animal was examined during the study period. Examinations included observations of general condition, skin and fur, eyes, nose, abdomen and external genitalia as well as an evaluation of respiration and observations for any unusual behavior or clinical signs of toxic or pharmacologic effects. The pharmacokinetic results are set forth in Table 2.

TABLE 2
in vivo Pharmacokinetics^{§} of PEG-Oligo conjugates

| | Cmax | T_{1/2} | MRT | CL | AUC |
|---|---|---|---|---|---|
| Compound | (μg/mL) | (hour) | (hour) | (mL/hour/log) | (μg/mL · hour) |
| Native siRNA | ND | ND | ND | ND | ND. |
| Compound 3 | 1018 ± 92 | 0.31 ± 0.05 | 0.45 ± 0.08 | 109 ± 13 | 460 ± 54.5 |
| Compound 5 | 97.7 ± 5.7 | 0.16 ± 0.01 | 0.23 ± 0.02 | 1128 ± 59 | 22.2 ± 1.2 |
| Compound 7 | 788 ± 53 | 0.32 ± 0.04 | 0.47 ± 0.06 | 136 ± 12 | 367 ± 33.0 |

^{§}PK estimates determined using a single compartment, iv bolus, first order elimination model
Mean ± SE; ND not detectable

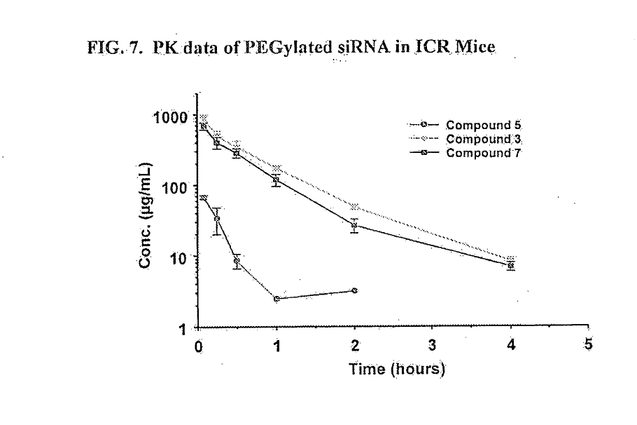

The plasma concentration-time curves of PEG-siRNA conjugates administered intravenously to mice are shown in FIG. 7. The mice tested with the native siRNA and compound 5 were dosed at 25 mg/kg siRNA equivalents. The mice tested with compound 3 and compound 7 received 50 mg/kg siRNA equivalents. In mice treated with the unconjugated native siRNA, the siRNA was undetectable in plasma within 5 minutes of administration. The results show that the siRNA conjugates significantly prolonged circulation of siRNA compared to native siRNA.

Without being bound by any theory, the pharmacodynamic properties of siRNA such as the biostability of siRNA were improved through enhanced siRNA resistance to degradation, i.e. nucleases.

### Example 9

### In Vivo Efficacy Study in Mice Xenografted with Human Lung Tumor Cells

BCL2 down regulation efficacy of the PEG-siRNA conjugates (compounds 3, 5 and 7) prepared using different linkers was evaluated in Nu/nu mice, Harlan Sprague-Dawley (female). The study also included the control group of mice injected with saline or native siRNA. H460 human non small cell lung tumors were established in nude mice by subcutaneous injection of 2.5×10⁶ cells/mouse into the right axillary flank. Tumor growth was monitored twice weekly and measured once palpable. When tumors reached an average volume of 70-80 mm³, the mice were divided into experimental groups (10/group). The tumor volume for each mouse was determined by measuring two dimensions with calipers and calculated using the formula: tumor volume=(length×width)/²).

Solutions containing each test compound were prepared insaline. Injection volume was 0.2 mL. Dosing solutions were made just prior to injection. Each test compound (compounds 3, 5 and 7) was injected intraperitoneally except that the native siRNA injected intratumorly. Each test compound was injected at 10 mg/kg (siRNA equivalent) once a day until the termination of the study. The first day of dosing is designated as Day 1. On day 9, three mice from each test group were sacrificed to study the BCL2 levels in the tumors. Most of the tumors from the animals were excised when the animals were sacrificed. Some tumors were not excised due to large ulcerations preventing a tumor sample from being taken. BCL2 mRNA levels for these tissues were quantified using RT-PCR and the results are set forth in Table 3.

TABLE 3
BCL2 down-modulation in vivo.

| | BCL2/18s rRNA | |
|---|---|---|
| Compound | log(Mean ± SD) | % Inhibition |
| Saline | 0.87 ± 0.15 | 0 |
| Native siRNA | 0.13 ± 0.06 | 81 |
| Compound 3 | 0.60 ± 0.20 | 47 |
| Compound 5 | 0.70 ± 0.10 | 23 |
| Compound 7 | 0.30 ± 0.17 | 73 |

The PEG-siRNA conjugates including bicine-based releasable linkers significantly dawn-regulated BCL2 mRNA expression in the tissues of mice xenografted with H460 human cancer cells. The conjugates according to the present invention allow cellular uptake of siRNA and mRNA down regulation in cancer cells in the absence of transfection agents. This technology benefits in vivo administration of therapeutic siRNA.

What is claimed is:

1. A siRNA conjugate of the formula (I):
A-R₁—(R₂)ₑ—R₃
wherein:
A is a capping group or
R′₃—(R′₂)_{e′}—;
R₁ is a substantially non-antigenic water-soluble polymer;
R₂ and R′₂ are independently selected releasable or permanent linkers or a combination thereof;
R₃ and R′₃ are the same or different siRNA-containing moiety; and
(e) and (e′) are the same or different positive integers.

2. The conjugate of claim 1, wherein R₂ is linked to the sense strand of the siRNA-containing moiety.

3. The conjugate of claim 1, wherein A is selected from the group consisting of H, NH₂, OH, CO₂H, C₁₋₆ alkoxy and C₁₋₆ alkyl.

4. A conjugate of claim 1 having a formula:
R′₃—(R′₂)_{e′}—R₁—(R₂)ₑ—R₃

5. The conjugate of claim 1, wherein R₂ and R′₂ are independently selected from the group consisting of benzyl elimination-based linkers, trialkyl lock-based linkers, bicine-based linkers, acid labile linkers, lysosomal cleavable peptides and capthepsin B cleavable peptides.

6. The conjugate of claim 1, wherein R₂ and R′₂ are independently selected from the group consisting of: 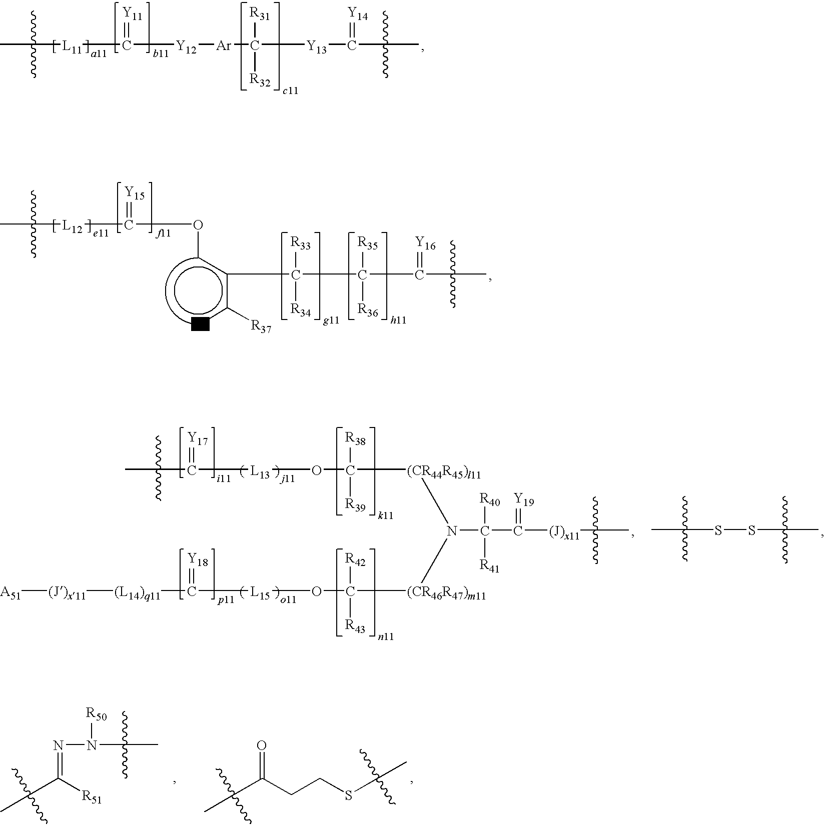
-Val-Cit-,
-Gly-Phe-Leu-Gly-,
-Ala-Leu-Ala-Leu-,
-Phe-Lys-, 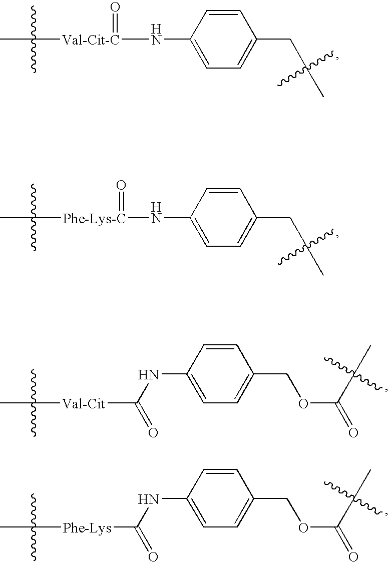
-Val-Cit-C(═O)—CH₂OCH₂—C(═O)—,
-Val-Cit-C(═O)—CH₂SCH₂—C(═O)—,
—NHCH(CH₃)—C(═O)—NH(CH₂)₆—C(CH₃₎₂—C(═O)—, and
—CH═N—NH—,
wherein,
Y₁₁₋₁₉ are independently O, S or NR₄₈;
R₃₁₋₄₈, R₅₀₋₅₁ and A₅₁ are independently selected from the group consisting of hydrogen, C₁₋₆ alkyls, C₃₋₁₂ branched alkyls, C₃₋₈ cycloalkyls, C₁₋₆substituted alkyls, C₃₋₈ substituted cyloalkyls, aryls, substituted aryls, aralkyls, C₁₋₆ heteroalkyls, substituted C₁₋₆ heteroalkyls, C₁₋₆ alkoxy, phenoxy and C₁₋₆ heteroalkoxy;
Ar is an aryl or heteroaryl moiety;
L₁₁₋₁₅ are independently selected bifunctional spacers;
J and J′ are independently moieties actively transported into a target cell or 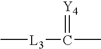
wherein
L₃ is a bifunctional linker;
Y₄ is O, S or NR₁₁; and
R₁₁ is selected from the group consisting of hydrogen, C₁₋₆ alkyls, C₃₋₁₂ branched alkyls, C₃₋₈ cycloalkyls, C₁₋₆ substituted alkyls, C₃₋₈ substituted cyloalkyls, aryls, substituted aryls, aralkyls, C₁₋₆ heteroalkyls, substituted C₁₋₆ heteroalkyls, C₁₋₆ alkoxy, phenoxy and C₁₋₆ heteroalkoxy;
(c11), (h11), (k11), (l11), (m11) and (n11) are independently selected positive integers;
(a11), (e11), (g11), (j11), (o11) and (q11) are independently: either zero or a positive integer; and
(b11), (x11), (x′11), (f11), (i11) and (p11) are independently zero or one.

7. The conjugate of claim 1, wherein R₂ and R′₂ are independently selected from the group consisting of
—[C(═O)]ᵥ(CR₂₂R₂₃)ₜ[C(═O)]_{v′}—,
—[C(═O)]ᵥ(CR₂₂R₂₃)ₜ—O[C(═O)]_{v′}—,
—[C(═O)]ᵥ(CR₂₂R₂₃)ₜ—NR₂₆[C(═O)]_{v′}—,
—[C(═O)]ᵥO(CR₂₂R₂₃)ₜ[C(═O)]_{v′}—,
—[C(═O)]ᵥO(CR₂₂R₂₃)ₜO[C(═O)]_{v′}—,
—[C(═O)]ᵥO(CR₂₂R₂₃)ₜNR₂₆[C(═O)]_{v′}—,
—[C(═O)]ᵥNR₂₁(CR₂₂R₂₃)ₜ[C(═O)]_{v′}—,
—[C(═O)]ᵥNR₂₁(CR₂₂R₂₃)ₜO[C(═O)]_{v′}—,
—[C(═O)]ᵥNR₂₁(CR₂₂R₂₃)ₜNR₂₆[C(═O)]_{v′}—,
—[C(═O)]ᵥ(CR₂₂R₂₃)ₜO—(CR₂₈R₂₉)_{t′}[C(═O)]_{v′}—,
—[C(═O)]ᵥ(CR₂₂R₂₃)ₜNR₂₆—(CR₂₈R₂₉)_{t′}[C(═O)]_{v′}—,
—[C(═O)]ᵥ(CR₂₂R₂₃)ₜS—(CR₂₈R₂₉)_{t′}[C(═O)]_{v′}—,
—[C(═O)]ᵥO(CR₂₂R₂₃)ₜO—(CR₂₈R₂₉)_{t′}[C(═O)]_{v′}—,
—[C(═O)]ᵥO(CR₂₂R₂₃)ₜNR₂₆—(CR₂₈R₂₉)_{t′}[C(═O)]_{v′}—,
—[C(═O)]ᵥO(CR₂₂R₂₃)ₜS—(CR₂₈R₂₉)_{t′}[C(═O)]_{v′}—,
—[C(═O)]ᵥNR₂₁(CR₂₂R₂₃)ₜO—(CR₂₈R₂₉)_{t′}[C(═O)]_{v′}—,
—[C(═O)]ᵥNR₂₁(CR₂₂R₂₃)ₜNR₂₆—(CR₂₈R₂₉)_{t′}[C(═O)]_{v′}—,
—[C(═O)]ᵥNR₂₁(CR₂₂R₂₃)ₜS—(CR₂₈R₂₉)_{t′}[C(═O)]_{v′}—,
—[C(═O)]ᵥ(CR₂₂R₂₃CR₂₈R₂₉O)ₜNR₂₆[C(═O)]_{v′}—,
—[C(═O)]ᵥ(CR₂₂R₂₃CR₂₈R₂₉O)ₜ[C(═O)]_{v′}—,
—[C(═O)]ᵥO(CR₂₂R₂₃CR₂₈R₂₉O)ₜNR₂₆[C(═O)]_{v′}—,
—[C(═O)]ᵥO(CR₂₂R₂₃CR₂₈R₂₉O)ₜ[C(═O)]_{v′}—,
—[C(═O)]ᵥNR₂₁(CR₂₂R₂₃CR₂₈R₂₉O)ₜNR₂₆[C(═O)]_{v′}—,
—[C(═O)]ᵥNR₂₁(CR₂₂R₂₃CR₂₈R₂₉O)ₜ[C(═O)]_{v′}—,
—[C(═O)]ᵥ(CR₂₂R₂₃CR₂₈R₂₉O)ₜ(CR₂₄R₂₅)_{t′}[C(═O)]_{v′}—,
—[C(═O)]ᵥO(CR₂₂R₂₃CR₂₈R₂₉O)ₜ(CR₂₄R₂₅)_{t′}[C(═O)]_{v′}—,
—[C(═O)]ᵥNR₂₁(CR₂₂R₂₃CR₂₈R₂₉O)ₜ(CR₂₄R₂₅)_{t′}[C(═O)]_{v′}—,
—[C(═O)]ᵥ(CR₂₂R₂₃CR₂₈R₂₉O)ₜ(CR₂₄R₂₅)_{t′}O[C(═O)]_{v′}—,
—[C(═O)]ᵥ(CR₂₂R₂₃)ₜ(CR₂₄R₂₅R₂₈R₂₉O)_{t′}[C(═O)]_{v′}—,
—[C(═O)]ᵥ(CR₂₂R₂₃)ₜ(CR₂₄R₂₅R₂₈R₂₉O)_{t′}NR₂₆[C(═O)]_{v′}—,
—[C(═O)]ᵥO(CR₂₂R₂₃CR₂₈R₂₉O)ₜ(CR₂₄R₂₅)_{t′}O[C(═O)]_{v′}—,
—[C(═O)]ᵥO(CR₂₂R₂₃)ₜ(CR₂₄R₂₅R₂₈R₂₉O)_{t′}[C(═O)]_{v′}—,
—[C(═O)]ᵥO(CR₂₂R₂₃)ₜ(CR₂₄R₂₅R₂₈R₂₉O)_{t′}NR₂₆[C(═O)]_{v′}—,
—[C(═O)]ᵥNR₂₁(CR₂₂R₂₃CR₂₈R₂₉O)ₜ(CR₂₄R₂₅)_{t′}O[C(═O)]_{v′}—,
—[C(═O)]ᵥNR₂₁(CR₂₂R₂₃)ₜ(CR₂₄R₂₅R₂₈R₂₉O)_{t′}[C(═O)]_{v′}—,
—[C(═O)]ᵥNR₂₁(CR₂₂R₂₃)ₜ(CR₂₄R₂₅R₂₈R₂₉O)_{t′}NR₂₆[C(═O)]_{v′}—, 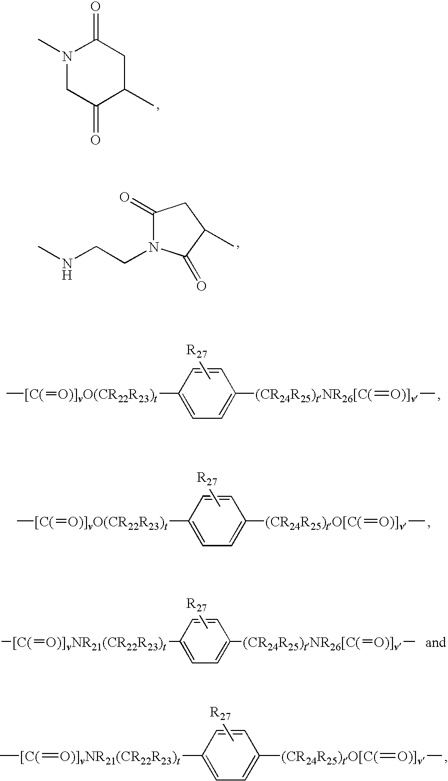
wherein
R₂₁₋₂₉ are independently, selected from the group consisting of hydrogen, C₁₋₆ alkyls, C₃₋₁₂ branched alkyls, C₃₋₈ cycloalkyls, C₁₋₆ substituted alkyls, C₃₋₈ substituted cyloalkyls, aryls, substituted aryls, aralkyls, C₁₋₆ heteroalkyls, substituted C₁₋₆ heteroalkyls, C₁₋₆ alkoxy, phenoxy and C₁₋₆ heteroalkoxy;
(t) and (t′) are independently zero or a positive integer, preferably zero or an integer; and
(v) and (v′) are independently zero or 1.

8. The conjugate of claim 1, wherein R₂ and R′₂ are independently selected amino acids or amino acid derivatives.

9. The conjugate of claim 1, wherein R₂ and R′₂ are independently selected from the group consisting of

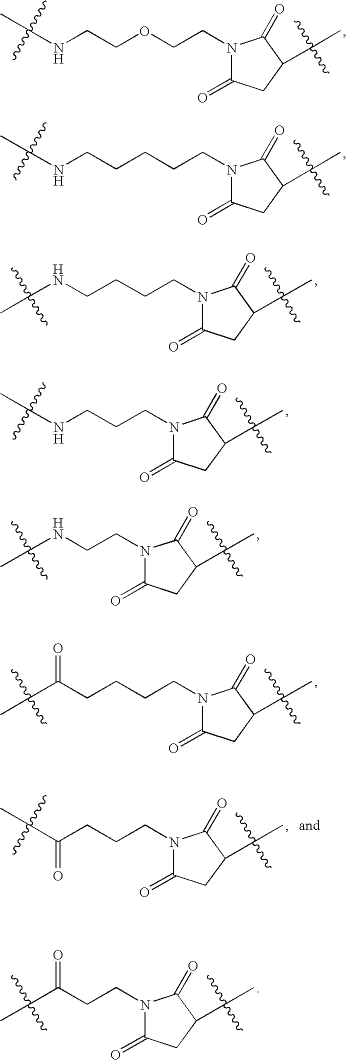

10. The conjugate of claim 1, wherein (e) and (e′) are independently 1 or 2.

11. The Conjugate of claim 1, wherein R₁ comprises a linear, terminally branched or multi-armed polyalkylene oxide.

12. The conjugate of claim 11, wherein the polyalkylene oxide is selected from the group consisting of polyethylene glycol and polypropylene glycol.

13. The conjugate of claim 11, wherein the polyalkylene oxide is selected from the group consisting of
—Y₇₁—(CH₂CH₂O)ₙ—CH₂CH₂Y₇₁—,
—Y₇₁—(CH₂CH₂O)ₙCH₂C(═Y₂₂)—Y₇₁—,
—Y₇₁—C(═Y₇₂)—(CH₂)ₐ₂—Y₇₃—(CH₂CH₂O)ₙ—CH₂CH₂—Y₇₃—(CH₂)ₐ₂—C(═Y₇₂)—Y₇₁— and
—Y₇₁—(CR₇₁R₇₂)ₐ₂—Y₇₃—(CH₂)_{b2}—O—(CH₂CH₂O)ₙ—(CH₂)_{b2}—Y₇₃—(CR₇₁R₇₂)ₐ₂—Y₇₁—,
wherein:
Y₇₁ and Y₇₃ are independently O, S, SO, SO₂, NR₇₃ or a bond;
Y₇₂ is O, S, or NR₇₄;
R₇₁₋₇₄ are independently selected from the group consisting of hydrogen C₁₋₆ alkyl, C₂₋₆ alkenyl, C₂₋₆ alkynyl, C₃₋₁₉ branched alkyl, C₃₋₈ cycloalkyl, C₁₋₆ substituted alkyl, C₂₋₆ substituted alkenyl, C₂₋₆ substituted alkynyl, C₃₋₈ substituted cycloalkyl, aryl, substituted aryl, heteroaryl, substituted heteroaryl, C₁₋₆ heteroalkyl, substituted C₁₋₆ heteroalkyl, C₁₋₆ alkoxy, aryloxy, C₁₋₆ heteroalkoxy, heteroaryloxy, C₂₋₆ alkanoyl, arylcarbonyl, C₂₋₆ alkoxycarbonyl, aryloxycarbonyl, C₂₋₆ alkanoyloxy, arylcarbonyloxy, C₂₋₆ substituted alkanoyl, substituted arylcarbonyl, C₂₋₆ substituted alkanoyloxy, substituted aryloxycarbonyl, C₂₋₆ substituted alkanoyloxy and substituted aryl carbonyloxy;
(a2) and (b2) are independently zero or a positive integer; and
(n) is an integer from about 10 to about 2300.

14. The conjugate of claim 11, wherein the polyalkylene oxide is a polyethylene glycol of the formula, —O—(CH₂CH₂O)ₙ—
wherein (n) is an integer from about 10 to about 2,300.

15. The conjugate of claim 1, wherein R₁ has an average molecular weight of from about 2,000 to about 100,000 daltons.

17. The conjugate of claim 1, wherein R₁ has an average molecular weight of from about 5,000 to about 60,000 daltons.

18. The conjugate of claim 1, wherein R₁ has an average molecular weight of from about 20,000 to about 45,000 daltons.

19. The conjugate of claim 1, wherein the antisense strand of the siRNA-containing moiety comprises about 18 to about 28 nucleotides complementary to a target gene.

20. The conjugate of claim 1, wherein the antisense strand of the siRNA-containing moiety comprises about 18 to about 28 nucleotides complementary to the nucleic acid sequence of SEQ ID NO: 1.

21. The conjugate of claim 1, wherein the antisense strand of the siRNA-containing moiety comprises the nucleic acid sequence of SEQ ID NO: 3.

22. A conjugate of claim 1 selected from the group consisting of: 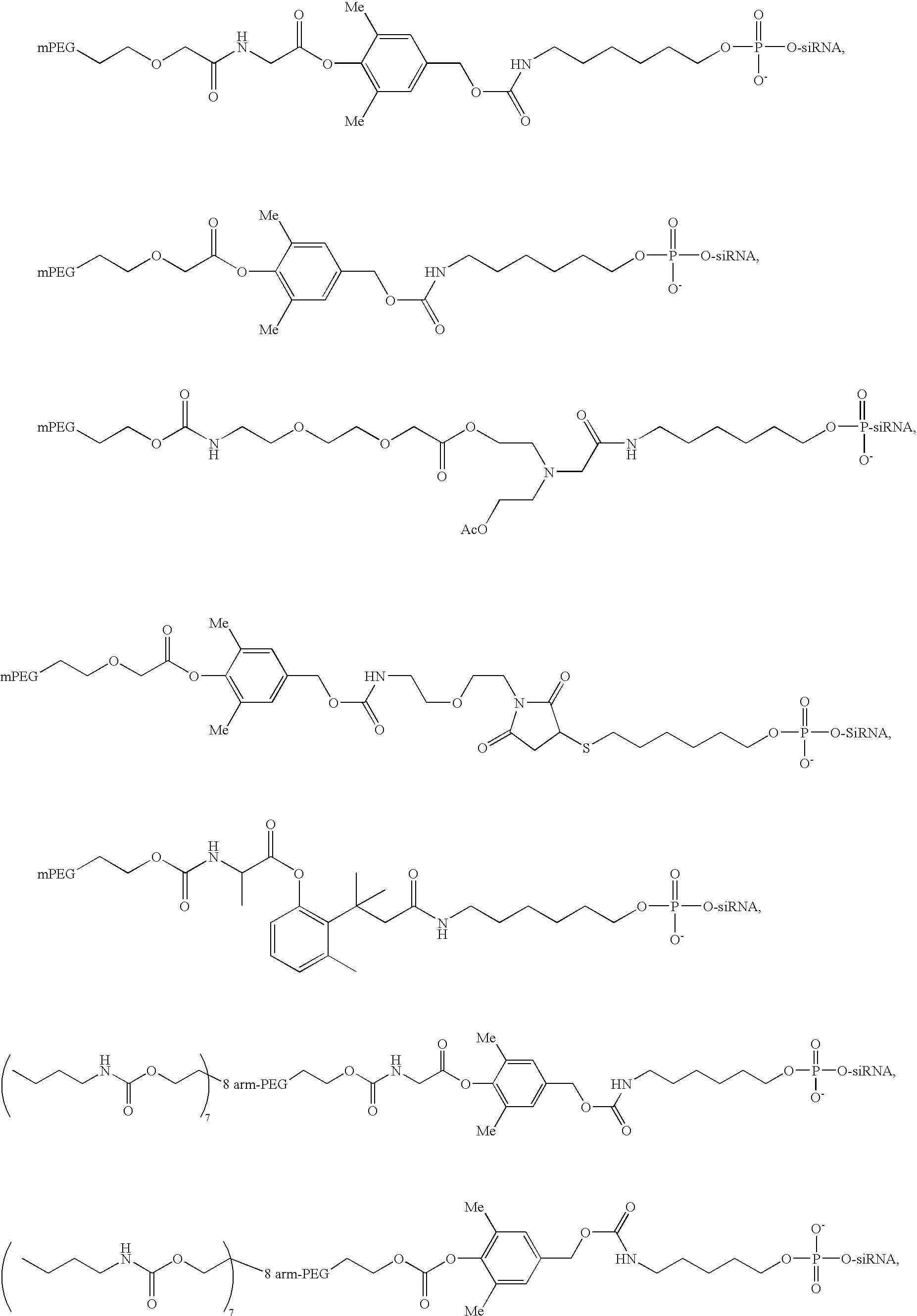 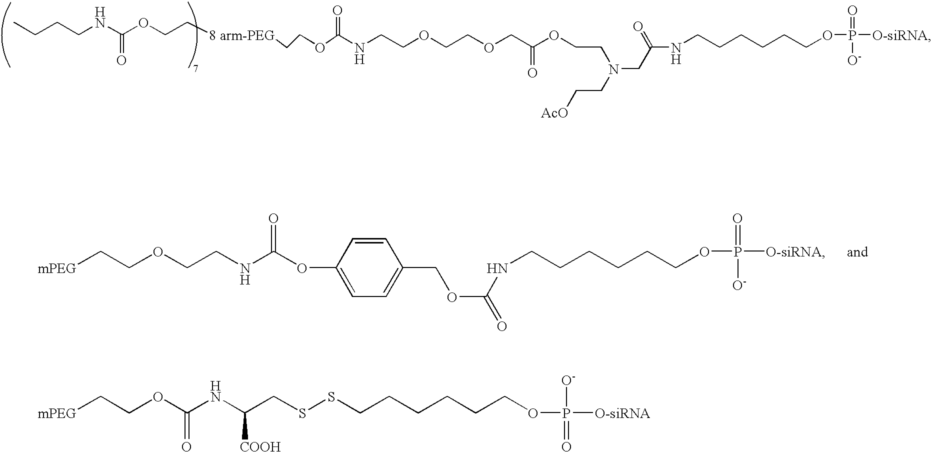
wherein the sense strand of the siRNA-containing moiety is conjugated to the polymer.

23. A conjugate of claim 1 selected from the group consisting of: 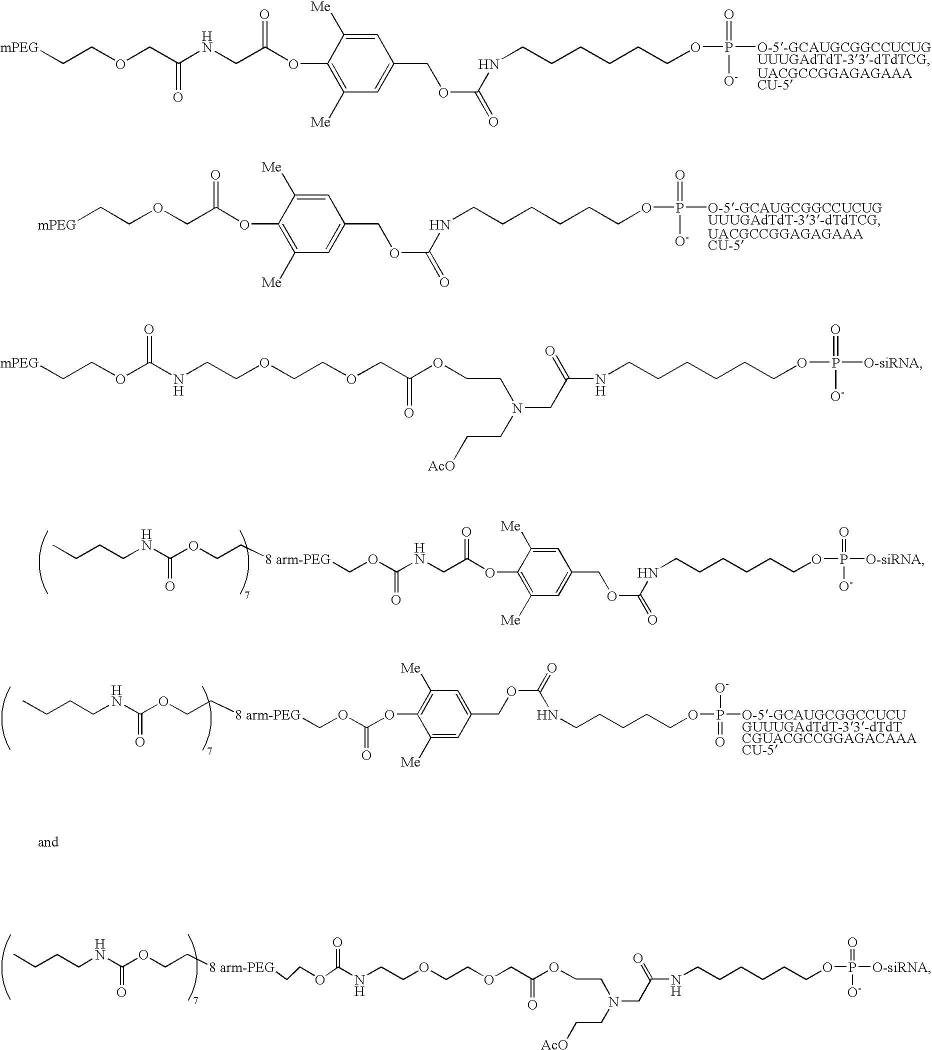
wherein
siRNA includes the nucleic acid sequences of SEQ ID NOs: 2 or 3; and
the 5′-end of the sense strand of the siRNA is modified to a C6-amino tail for conjugating to PEG linkers.

24. The conjugate of claim 5, wherein the acid labile linker is selected from the group consisting of a disulfide linker, hydrazone-containing linkers and thiopropionate-containing linkers.

25. A method of inhibiting a gene expression in human cells or tissues, comprising contacting human, cells or tissues with a conjugate of claim 1.

26. The method of claim 25, wherein the cells or tissues are cancer cells or tissues.

27. The method of claim 26, further comprising contacting the cells or tissues with a chemotherapeutic agent.

28. The method of claim 25, wherein the expression of BCL2 is inhibited.

29. The method of claim 28, wherein the antisense strand of the siRNA-containing moiety comprises about 18 to about 28 nucleotides complementary to the nucleic acid sequence of SEQ ID NO: 1.

30. The method of claim 28, wherein the conjugate is selected from the group consisting of

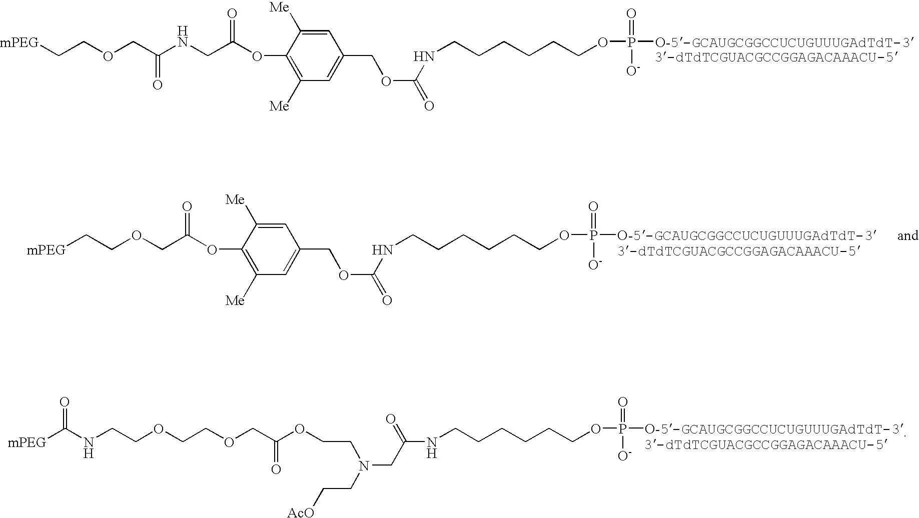

31. A method of inhibiting the growth or proliferation of cancer cells, comprising cancer cells with a conjugate of claim 1.

32. The method of claim 31, wherein the antisense strand of the siRNA-containing moiety comprises about 18 to about 28 nucleotides complementary to the nucleic acid sequence of SEQ ID NO: 1.

33. The method of claim 31, wherein the conjugate is selected from the group consisting of [chemical structure]


SEQUENCE LISTING (3 sequences)

SEQ ID NO: 1 - DNA, 5086 bp, Homo sapiens
gcgcccgcccctccgcgccgcctgcccgcccgcccgccgcgctcccgcccgccgctctccgtggccccgccgcgctgccgccgccgccgctgccagcgaaggtgccggggctccgggccctccctgccggcggccgtcagcgctcggagcgaactgcgcgacgggaggtccgggaggcgaccgtagtcgcgccgccgcgcaggaccaggaggaggagaaagggtgcgcagcccggaggcggggtgcgccggtggggtgcagcggaagagggggtccaggggggagaacttcgtagcagtcatcctttttaggaaaagagggaaaaaataaaaccctcccccaccacctccttctccccacccctcgccgcaccacacacagcgcgggcttctagcgctcggcaccggcgggccaggcgcgtcctgccttcatttatccagcagcttttcggaaaatgcatttgctgttcggagtttaatcagaagacgattcctgcctccgtccccggctccttcatcgtcccatctcccctgtctctctcctggggaggcgtgaagcggtcccgtggatagagattcatgcctgtgtccgcgcgtgtgtgcgcgcgtataaattgccgagaaggggaaaacatcacaggacttctgcgaataccggactgaaaattgtaattcatctgccgccgccgctgccaaaaaaaaactcgagctcttgagatctccggttgggattcctgcggattgacatttctgtgaagcagaagtctgggaatcgatctggaaatcctcctaatttttactccctctccccccgactcctgattcattgggaagtttcaaatcagctataactggagagtgctgaagattgatgggatcgttgccttatgcatttgttttggttttacaaaaaggaaacttgacagaggatcatgctgtacttaaaaaatacaagtaagtctcgcacaggaaattggtttaatgtaactttcaatggaaacctttgagattttttacttaaagtgcattcgagtaaatttaatttccaggcagcttaatacattgtttttagccgtgttacttgtagtgtgtatgccctgctttcactcagtgtgtacagggaaacgcacctgattttttacttattagtttgttttttctttaacctttcagcatcacagaggaagtagactgatattaacaatacttactaataataacgtgcctcatgaaataaagatccgaaaggaattggaataaaaatttcctgcgtctcatgccaagagggaaacaccagaatcaagtgttccgcgtgattgaagacaccccctcgtccaagaatgcaaagcacatccaataaaatagctggattataactcctcttctttctctgggggccgtggggtgggagctggggcgagaggtgccgttggcccccgttgcttttcctctgggaaggatggcgcacgctgggagaacggggtacgacaaccgggagatagtgatgaagtacatccattataagctgtcgcagaggggctacgagtgggatgcgggagatgtgggcgccgcgcccccgggggccgcccccgcaccgggcatcttctcctcccagcccgggcacacgccccatccagccgcatcccgcgacccggtcgccaggacctcgccgctgcagaccccggctgcccccggcgccgccgcggggcctgcgctcagcccggtgccacctgtggtccacctggccctccgccaagccggcgacgacttctcccgccgctaccgcggcgacttcgccgagatgtccagccagctgcacctgacgcccttcaccgcgcggggacgctttgccacggtggtggaggagctcttcagggacggggtgaactgggggaggattgtggccttctttgagttcggtggggtcatgtgtgtggagagcgtcaaccgggagatgtcgcccctggtggacaacatcgccctgtggatgactgagtacctgaaccggcacctgcacacctggatccaggataacggaggctgggatgcctttgtggaactgtacggccccagcatgcggcctctgtttgatttctcctggctgtctctgaagactctgctcagtttggccctggtgggagcttgcatcaccctgggtgcctatctgagccacaagtgaagtcaacatgcctgccccaaacaaatatgcaaaaggttcactaaagcagtagaaataatatgcattgtcagtgatgtaccatgaaacaaagctgcaggctgtttaagaaaaaataacacacatataaacatcacacacacagacagacacacacacacacaacaattaacagtcttcaggcaaaacgtcgaatcagctatttactgccaaagggaaatatcatttattttttacattattaagaaaaaagatttatttatttaagacagtcccatcaaaactccgtctttggaaatccgaccactaattgccaaacaccgcttcgtgtggctccacctggatgttctgtgcctgtaaacatagattcgctttccatgttgttggccggatcaccatctgaagagcagacggatggaaaaaggacctgatcattggggaagctggctttctggctgctggaggctggggagaaggtgttcattcacttgcatttctttgccctgggggcgtgatattaacagagggagggttcccgtggggggaagtccatgcctccctggcctgaagaagagactctttgcatatgactcacatgatgcatacctggtgggaggaaaagagttgggaacttcagatggacctagtacccactgagatttccacgccgaaggacagcgatgggaaaaatgcccttaaatcataggaaagtatttttttaagctaccaattgtgccgagaaaagcattttagcaatttatacaatatcatccagtaccttaaaccctgattgtgtatattcatatattttggatacgcaccccccaactcccaatactggctctgtctgagtaagaaacagaatcctctggaacttgaggaagtgaacatttcggtgacttccgatcaggaaggctagagttacccagagcatcaggccgccacaagtgcctgcttttaggagaccgaagtccgcagaacctacctgtgtcccagcttggaggcctggtcctggaactgagccgggccctcactggcctcctccagggatgatcaacagggtagtgtggtctccgaatgtctggaagctgatggatggagctcagaattccactgtcaagaaagagcagtagaggggtgtggctgggcctgtcaccctggggccctccaggtaggcccgttttcacgtggagcataggagccacgacccttcttaagacatgtatcactgtagagggaaggaacagaggccctgggccttcctatcagaaggacatggtgaaggctgggaacgtgaggagaggcaatggccacggcccattttggctgtagcacatggcacgttggctgtgtggccttggccacctgtgagtttaaagcaaggctttaaatgactttggagagggtcacaaatcctaaaagaagcattgaagtgaggtgtcatggattaattgacccctgtctatggaattacatgtaaaacattatcttgtcactgtagtttggttttatttgaaaacctgacaaaaaaaaagttccaggtgtggaatatgggggttatctgtacatcctggggcattaaaaaaaaatcaatggtggggaactataaagaagtaacaaaagaagtgacatcttcagcaaataaactaggaaatttttttttcttccagtttagaatcagccttgaaacattgatggaataactctgtggcattattgcattatataccatttatctgtattaactttggaatgtactctgttcaatgtttaatgctgtggttgatatttcgaaagctgctttaaaaaaatacatgcatctcagcgtttttttgtttttaattgtatttagttatggcctatacactatttgtgagcaaaggtgatcgttttctgtttgagatttttatctcttgattcttcaaaagcattctgagaaggtgagataagccctgagtctcagctacctaagaaaaacctggatgtcactggccactgaggagctttgtttcaaccaagtcatgtgcatttccacgtcaacagaattgtttattgtgacagttatatctgttgtccctttgaccttgtttcttgaaggtttcctcgtccctgggcaattccgcatttaattcatggtattcaggattacatgcatgtttggttaaacccatgagattcattcagttaaaaatccagatggcgaatgaccagcagattcaaatctatggtggtttgacctttagagagttgctttacgtggcctgtttcaacacagacccacccagagccctcctgccctccttccgcgggggctttctcatggctgtccttcagggtcttcctgaaatgcagtggtcgttacgctccaccaagaaagcaggaaacctgtggtatgaagccagacctccccggcgggcctcagggaacagaatgatcagacctttgaatgattctaatttttaagcaaaatattattttatgaaaggtttacattgtcaaagtgatgaatatggaatatccaatcctgtgctgctatcctgccaaaatcattttaatggagtcagtttgcagtatgctccacgtggtaagatcctccaagctgctttagaagtaacaatgaagaacgtggacgtttttaatataaagcctgttttgtcttttgttgttgttcaaacgggattcacagagtatttgaaaaatgtatatatattaagaggtcacgggggctaattgctagctggctgccttttgctgtggggttttgttacctggttttaataacagtaaatgtgcccagcctcttggccccagaactgtacagtattgtggctgcacttgctctaagagtagttgatgttgcattttccttattgttaaaaacatgttagaagcaatgaatgtatataaaagc

SEQ ID NO: 2 - DNA, 21 bp, Artificial Sequence
  Description of Combined DNA/RNA Molecule Synthetic oligonucleotide
gcaugcggccucuguuugatt

SEQ ID NO: 3 - DNA, 21 bp, Artificial Sequence
  Description of Combined DNA/RNA Molecule Synthetic oligonucleotide
ucaaacagaggccgcaugctt